(19) United States
(12) Patent Application Publication
(10) Pub. No.: US 2024/0200100 A1
(43) Pub. Date: Jun. 20, 2024

(54) PACKAGING CELLS WITH TARGETED GENE KNOCKOUTS THAT IMPROVE RETROVIRAL VECTOR TITERS

(71) Applicant: The Regents of the University of California, Oakland, CA (US)

(72) Inventors: Donald B. Kohn, Tarzana, CA (US); Roger Paul Hollis, Los Angeles, CA (US); Jiaying Han, Los Angeles, CA (US)

(73) Assignee: The Regents of the University of California, Oakland, CA (US)

(21) Appl. No.: 18/555,508

(22) PCT Filed: Apr. 19, 2022
(86) PCT No.: PCT/US2022/025403

§ 371 Date: Oct. 13, 2023

Related U.S. Application Data
(60) Provisional application No. 63/177,300, filed on Apr. 20, 2021.

(51) Int. Cl. C12N 15/86; C12N 5/071; C12N 9/22; C12N 15/11; C12N 15/90

(52) U.S. Cl. CPC C12N 15/86; C12N 5/0686; C12N 9/22; C12N 15/11; C12N 15/907; C12N 2310/20; C12N 2740/15043; C12N 2740/15052; C12N 2800/80

(57) ABSTRACT

In various embodiments packaging cells are provided that increase provide increased titer and are useful for lentiviral production. In certain embodiments the packaging cells comprise a knockout of one or more genes selected from the group consisting of IFNAR1, ATR, OAS1, LDLR, and PKR.

### CROSS-REFERENCE TO RELATED APPLICATIONS

This application claims benefit of, and priority to U.S. Ser. No. 63/177,300, filed on Apr. 20, 2021, which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS A TEXT FILE

[Not Applicable]

### BACKGROUND

Retroviruses are enveloped RNA viruses that, after infection of a host cell, reverse transcribe their RNA genomes into a DNA intermediate, or provirus. All viruses containing an RNA genome and producing an RNA-dependent DNA polymerase are contained in the retroviral family. The family is divided into three subfamilies: (1) Oncovirinae, including all the oncogenic retroviruses, and several closely related non-oncogenic viruses; (2) Lentivirinae, the “slow retroviruses” such as the human immunodeficiency virus (HIV) and visna virus; and (3) Spumavirinae, the “foamy” retroviruses that induce persistent infections, generally without causing any clinical disease.

Retroviruses contain at least three types of proteins encoded by the viral genome, i.e., gag proteins (the group antigen internal structural proteins), pol proteins (the RNA-dependent DNA polymerase and the protease and integrase proteins), and env proteins (the viral envelope protein or proteins). In addition to genes encoding the gag, pol, and env proteins, the genome to the retrovirus includes two long terminal repeat (LTR) sequences, one at the 5′ and one at the 3′ end of the virus. These 5′ and 3′ LTRs promote transcription and polyadenylation of viral mRNAs and participate in the integration of the viral genome into the cellular DNA of the host.

The provirus can be stably integrated into the host's cellular DNA. Gene products encoded by the provirus are then expressed by the host cell to produce retroviral virions, thereby replicating the virus. Because the retroviral genome can be manipulated to include exogenous nucleotide sequence(s) of interest for expression in a target cell, retroviral vectors are important tools for stable gene transfer into mammalian cells.

Many proposed gene therapy applications use retroviral vectors to take advantage of the ability of these naturally infectious agents to transfer and efficiently express recombinant nucleotide sequences in susceptible target cells. Retroviral vectors suitable for use in such applications are generally defective retroviral vector that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, and/or in pol (see, e.g., Coffin, J., In: RNA Tumor Viruses, Weiss, R. et al., (ed) Cold Spring Harbor Laboratory, Vol. 2, pp. 36-73, 1985).

Retroviral vectors and packaging cells (helper cells) have been developed to introduce recombinant nucleic acid molecules into mammalian cells without the danger of the production of replicating infectious virus. This methodology uses two components, a retroviral vector and a packaging cell. The retroviral vector contains long terminal repeats (LTRs), the foreign DNA to be transferred, and a packaging sequence. This retroviral vector will not reproduce by itself because the genes that encode the structural and envelope proteins are not included within the vector. The packaging cell contains genes encoding the gag, pol, and env proteins, but does not contain the packaging signal, so that the cell can only form empty virus particles by itself. With this method, the retroviral vector is introduced into the packaging cell, to create a cell able to produce virus. The cell manufactures viral particles containing only the retroviral vector DNA, and therefore has been considered safe.

In multiple clinical trials, autologous hematopoietic stem cell (HSCs) transplantation in combination with gene therapy has produced clinical benefits with desirable long-term engraftment of gene corrected HSC and stable gene expression. Despite these early successes, the lengthy and complex nature of some lentiviral vectors (LV) has led to several challenges for clinical translation. It has been observed that titers of LVs decrease substantially with increasing proviral length. The reduction in titer, especially in the vectors with complex expression cassettes, creates a barrier for scaling up Good Manufacturing Practices (GMP)-grade vector production and significantly increases the production cost for clinical trials and commercialization.

Accordingly an increase in titer produced by packaging cells would greatly facilitate lentiviral production for clinical use.

### SUMMARY

Various embodiments provided herein may include, but need not be limited to, one or more of the following:

Embodiment 1: A recombinant retroviral packaging cell, said packaging cell comprising:
a mammalian cell wherein one or more mammalian genes that inhibit virus production are knocked out or knocked down.

Embodiment 2: The packaging cell of embodiment 1, wherein said cell is a mammalian cell modified to provide at least two packaging components for the surface or envelope of a retrovirus.

Embodiment 3: The packaging cell according to any one of embodiments 1-2, wherein said packaging cell is a cell modified to express retroviral Gag, Pol, and Env genes.

Embodiment 4: The packaging cell according to any one of embodiments 1-3, wherein said one or more mammalian genes comprises a gene that encodes a low-density lipoprotein receptor (LDLR) gene.

Embodiment 5: The packaging cell according to any one of embodiments 1-4, wherein said one or more mammalian genes that are knocked out or knocked down comprise one or more mammalian genes that regulates the DNA damage response pathway, and/or regulate transcription, and/or regulate innate immunity.

Embodiment 6: The packaging cell according to any one of embodiments 1-5, wherein said one or more mammalian genes that are knocked out or knocked down comprise a gene that mediates innate immunity.

Embodiment 7: The packaging cell of embodiment 6, herein said one or more mammalian genes that are knocked out or knocked down comprise a gene that is a member of the oligoadenylate synthetase (OAS) family.

Embodiment 8: The packaging cell of embodiment 7, wherein said herein said one or more mammalian genes that are knocked out or knocked down comprise OAS1.

Embodiment 9: The packaging cell of embodiment 7, wherein said herein said one or more mammalian genes that are knocked out or knocked down comprise OAS2.

Embodiment 10: The packaging cell of embodiment 7, wherein said herein said one or more mammalian genes that are knocked out or knocked down comprise OAS3.

Embodiment 11: The packaging cell of embodiment 7, wherein said herein said one or more mammalian genes that are knocked out or knocked down comprise OASL.

Embodiment 12: The packaging cell according to any one of embodiments 6-11, wherein said wherein said herein said one or more mammalian genes that are knocked out or knocked down comprise IFNAR1.

Embodiment 13: The packaging cell according to any one of embodiments 6-12, wherein said wherein said herein said one or more mammalian genes that are knocked out or knocked down comprise IFNAR2.

Embodiment 14: The packaging cell according to any one of embodiments 5-13, wherein said one or more mammalian genes comprises a gene that regulates transcription.

Embodiment 15: The packaging cell of embodiment 14, wherein said one or more mammalian genes that are knocked out or knocked down comprise the PKR gene.

Embodiment 16: The packaging cell according to any one of embodiments 5-15, wherein said one or more mammalian genes that are knocked out or knocked down comprise a gene that regulates a DNA damage response pathway.

Embodiment 17: The packaging cell of embodiment 16, wherein said one or more genes that are knocked out or knocked down comprise the PKR gene.

Embodiment 18: The packaging cell according to any one of embodiments 1-17, wherein said one or more mammalian genes are knocked down.

Embodiment 19: The packaging cell of embodiment 18, wherein ATR is knocked down and not knocked out.

Embodiment 20: The packaging cell according to any one of embodiments 1-17, wherein said one or more genes are knocked out.

Embodiment 21: The packaging cell according to any one of embodiments 1-5, wherein said one or more mammalian genes that are knocked out or knocked down comprise a member of the OAS gene family, PKR, and LDLR.

Embodiment 22: The packaging cell of embodiment 21, wherein said one or more mammalian genes that are knocked out or knocked down comprise one or more genes selected from the group consisting of 2′-5′-oligoadenylate synthetase 1 (OAS1), low-density lipoprotein receptor (LDLR), and PKR.

Embodiment 23: The packaging cell of embodiment 22, wherein said one or more mammalian genes that are knocked out or knocked down comprise OAS1.

Embodiment 24: The packaging cell according to any one of embodiments 22-23, wherein said one or more mammalian genes that are knocked out or knocked down comprise LDLR.

Embodiment 25: The packaging cell according to any one of embodiments 22-24, wherein said one or more mammalian genes that are knocked out or knocked down comprise PKR.

Embodiment 26: The packaging cell according to any one of embodiments 22-25, wherein said one or more mammalian genes are knocked out.

Embodiment 27: The packaging cell according to any one of embodiments 1-26, wherein said one or more mammalian genes are knocked out or knocked down using targeted CRISPR-mediated knockout.

Embodiment 28: The packaging cell of embodiment 27, wherein said one or more mammalian genes are knocked out using CRISPR/Cas.

Embodiment 29: The packaging cell according to any one of embodiments 1-26, wherein said one or more mammalian genes are knocked out or knocked down using RNAi.

Embodiment 30: The packaging cell of embodiment 29, wherein said one or more mammalian genes are knocked down using RNAi.

Embodiment 31: The packaging cell of embodiment 29, wherein said one or more mammalian genes are knocked out using RNAi.

Embodiment 32: The packaging cell according to any one of embodiments 1-31, wherein said cell is modified to express or to overexpress a transcription elongation factor.

Embodiment 33: The packaging cell of embodiment 32, wherein said cell is modified to express elongation factors SPT4 and/or SPT5.

Embodiment 34: The packaging cell of embodiment 33, wherein said cell is modified to express elongation factors SPT4 and SPT5.

Embodiment 35: The packaging cell according to any one of embodiments 33-37, wherein an expression cassette that expresses elongation factors SPT4 and/or SPT5 is episomal in said packaging cell.

Embodiment 36: The packaging cell according to any one of embodiments 33-37, wherein an expression cassette that expresses elongation factors SPT4 and/or SPT5 is integrated into the genome of said packaging cell.

Embodiment 37: The packaging cell according to any one of embodiments 32-36, wherein a nucleic acid encoding said elongation factors is operably linked to a constitutive promoter.

Embodiment 38: The packaging cell according to any one of embodiments 32-36, wherein a nucleic acid encoding said elongation factors is operably linked to an inducible promoter.

Embodiment 39: The packaging cell according to any one of embodiments 1-38, wherein said mammalian cell is selected from the group consisting of HEK293, HEK293T, TE671, HT1080, 3T3, K562, 3T3, U937, and H9.

Embodiment 40: The packaging cell of embodiment 39, wherein said cell is a HEK293T cell.

Embodiment 41: The packaging cell according to any one of embodiments 1-40, wherein said cell, when transfected with a defective recombinant retroviral genome, produces complete virion at a higher titer and/or infectivity than the same cell without said one or more mammalian genes knocked out and without transcription elongation factors overexpressed.

Embodiment 42: The packaging cell of embodiment 41, wherein said packaging cell increases lentiviral titer.

Embodiment 43: The packaging cell according to any one of embodiments 41-42, wherein said packing cells increase titer for complex lentiviral vectors.

Embodiment 44: The packaging cell according to any one of embodiments 41-43, wherein said packaging cells increase titer for LV in reverse orientation.

Embodiment 45: The packaging cell according to any one of embodiments 1-44, wherein said cell is transfected with a defective, recombinant retroviral genome containing a nucleotide sequence of interest.

Embodiment 46: The packaging cell of embodiment 45, wherein said cell is transfected with a lentiviral (LV) genome.

Embodiment 47: The packaging cell of embodiment 45, wherein said cell is transfected with an HIV lentiviral (LV) genome.

Embodiment 48: The packaging cell according to any one of embodiments 45-47, wherein said cell is transfected with a lentiviral (LV) genome comprising an expression cassette in reverse orientation.

Embodiment 49: The packaging cell according to any one of embodiments 45-48, wherein said gene of interest comprises gene or cDNA selected from the group consisting of βAS3, FOXP3, WAS, RAG1, CAR (chimeric antigen receptor), and TCR (T-cell receptor).

Embodiment 50: The packaging cell of embodiment 49, wherein said LV genome comprises βAS3-FB.

Embodiment 51: A method of producing a retrovirus vector, said method comprising:
culturing a packaging cell according to any one of embodiments 1-44, wherein said packaging cell is transfected with a defective, recombinant retroviral genome containing a nucleotide sequence of interest where said viral genome is packaged within a viral capsid within said cell to produce a virion; and
recovering and isolating said virion.

Embodiment 52: The method of embodiment 51, wherein said cell is transfected with a lentiviral (LV) genome.

Embodiment 53: The method of embodiment 51, wherein said cell is transfected with an HIV lentiviral (LV) genome.

Embodiment 54: The method according to any one of embodiments 51-53, wherein said gene of interest comprises gene or cDNA selected from the group consisting of βAS3, FOXP3, WAS, RAG1, CAR, and TCR.

### Definitions

A “defective recombinant retroviral genome” refers to a retroviral genome lacking or more genes necessary for encapsidaton and production of a complete viral particle (virion). In certain embodiments a defective retroviral genome lacks one or more of the viral Gag, Pol, and/or Env genes. In certain embodiments lacks all of the Gag, Pol, and Env genes.

As used herein, the term “packaging cell” refers to a cell that contains those elements necessary for production of infectious recombinant virus when transfected with a defective recombinant retroviral genome. Such packaging cells are capable of expressing viral structural proteins (such as gag-pol and env, which may be codon optimized) but they typically do not contain a packaging signal.

The term “packaging signal” which is referred to interchangeably as “packaging sequence” or “psi” is used in reference to a non-coding, cis-acting sequence required for encapsidation of retroviral RNA strands during viral particle formation. The psi sequence is well known to those of skill the art. It is noted for example, that in HIV-1, this sequence has been mapped to loci extending from upstream of the major splice donor site (SD) to at least the gag start codon

The term knockout” refers to the disruption of a gene that results in partial or complete suppression of the expression of at least a portion of a protein encoded by that gene and/or a reduction or elimination of activity of the polypeptide encoded by that gene, e.g., as compared to the same cell without the “knockout” (disruption). The knockout is typically the result of genomic disruptions, including transposons, tilling, and homologous recombination, antisense constructs, sense constructs, or targeted disruption of the gene using, for example, a zinc finger protein, TALEN, or a CRISPR/Cas construct.

The term knockdown” refers to a method of gene silencing responsible for the temporary inactivation of a particular gene product. It is typically applicable in the RNA level and it targets the mRNA produced by the transcription of the target gene. Therefore, gene knockdown is a form of post-transcriptional regulation of gene expression. In certain embodiments it is based on the RNA interference (RNAi) pathway by allowing the degradation of mRNA. Here, miRNA, siRNA, and shRNA play a key role by binding to the target mRNA. The resultant RNA duplexes are degraded by the action of Dicer and RISC. They will turn off the expression of the gene of interest temporally. In certain embodiments, however, a knockdown can readily be accomplished using a CRISPR/Cas system, e.g., to target the transcribed RNA. In general, gene knockout refers to a permanent change in DNA leading to the loss of function of a gene, caused by a manipulation of the organism's DNA while gene knockdown refers to a temporary decrease in gene expression.

The phrases “disruption of the gene” and “gene disruption” refer to the deletion or insertion of a nucleic acid sequence into one region of the native DNA sequence and/or the promoter region of a gene so as to decrease or prevent expression of that gene or to decrease or eliminate activity of a protein expressed by that gene in the cell as compared to the wild-type or naturally occurring sequence of the gene.

A “lentivirus” as used herein refers to a genus of the Retroviridae family. Lentiviruses are unique among the retroviruses in being able to infect non-dividing cells. They can deliver a significant amount of genetic information into the DNA of the host cell, so they are one of the most efficient methods of a gene delivery vector. HIV, SIV, and FIVE are all examples of lentiviruses. Vectors derived from lentiviruses offer the means to achieve significant levels of gene transfer in vivo.

A “complex vector” (e.g., a complex lentiviral vector) is a vector with long regulatory elements, such as enhancers and insulators, and/or a reverse-oriented internal promoter.

### BRIEF DESCRIPTION OF THE DRAWINGS

FIG. 1 shows that knocking out antiviral genes in HEK293T packaging cells increased lentiviral vector titers.

FIG. 2, panels A-C, shows that knocking out IFNAR1, ATR, OAS1, and LDLR in 293T cells increased titers. Panel (A IFNAR1, ATR, and OAS1 protein expression in parental and gene-edited cells measured by western blot. HEK293T cells were edited by CRISPR-Cas9 targeting each of the genes. The edited cells were sorted for single-cell clones, and an isogenic clone with no parental allele, except ATR, was expanded for protein expression analysis. Because ATR is an essential gene for cell survival, an ATR KD clone with 33% parental allele remaining was selected. β-Actin and Vinculin were used as the loading controls. Panel B) LDLR protein expression in parental, LDLR−/− 293T cells, and unstimulated CD34+ HSPCs measured by flow cytometry. Unstimulated CD34+ HSPCs are known to not express LDLR and were used as a negative control. A mouse IgG1 antibody was used as the isotype control. Panel C) Titers of Lenti/βAS3-FB packaged in parental and gene-edited cells (n=9-29 dishes of identical cultures from three to 10 independent experiments; bars represent mean with SD; unpaired t test, *p<0.05, **p<0.01, ***p<0.001, ****p<0.0001). PKR−/− cells are known to increase LV titers and were used as a positive control to evaluate titer increase. LVs in unconcentrated viral supernatant were assayed for titer by transducing HT29 cells at 10-fold serial dilution and VCN measured by ddPCR.

FIG. 3, panels A-E, shows that restoring protein expression of LDLR and OAS1 decreased titers. Panel A) LDLR protein expression in parental 293T, LDLR−/− 293T, LDLR−/− 293T transduced with Lenti/GFP, and LDLR−/− 293T transduced with Lenti/LDLR measured by flow cytometry. LDLR−/− cells were transduced with an LV encoding LDLR to restore its expression, or an LV encoding GFP as a transduction control. Panel B) Percentage of LDLR+ cells in parental, LDLR−/−, and LDLR restored cells measured by flow cytometry. Panel C) Titers of Lenti/βAS3-FB packaged in parental 293T, LDLR−/− 293T, LDLR−/− 293T transduced with Lenti/GFP, and LDLR−/− 293T transduced with Lenti/LDLR. Because Lenti/LDLR carries an ires-GFP cassette, all transduced cells were sorted for the GFP+ population. The sorted GFP+ cells were expanded for VCN analysis by ddPCR and packaging (n=9 dishes of identical cultures from three independent experiments; bars represent mean with SD; unpaired t test, *p<0.05, **p<0.01, ***p<0.001, ****p<0.0001). Panel D) OAS1 protein expression in parental 293T, OAS1−/− 293T, OAS1−/− 293T transduced with Lenti/GFP, and OAS1−/− 293T transduced with Lenti/OAS1 measured by western blot. Panel E) Titers of Lenti/βAS3-FB packaged in parental 293T, OAS1−/− 293T, OAS1−/− 293T transduced with Lenti/GFP, and OAS1−/− 293T transduced with Lenti/OAS1 (n=12 dishes of identical cultures from four independent experiments; bars represent mean with SD; unpaired t test, *p<0.05, **p<0.01, ***p<0.001, ****p<0.0001).

FIG. 4, panels A-E, shows that knocking out PKR, OAS1, and LDLR in 293T cells additively increased titer, RNA, and physical particles. Panel A) Fold difference of titers of Lenti/βAS3-FB packaged in single, double, and triple-KO cells (n=12 dishes of identical cultures from four independent experiments; bars represent mean with SD; unpaired t test, *p<0.05, **p<0.01, ***p<0.001, ****p<0.0001). The double-KO isogenic clones were created by electroporating RNP targeting the OAS1 gene in the PKR−/− isogenic clones and selecting an isogenic clone with OAS1 and PKR knocked out. The triple-KO isogenic clones were created by electroporating RNP targeting LDLR gene in the PKR and OAS1 double-KO clone and selecting an isogenic clone with OAS1, PKR, and LDLR knocked out. Panels B and C) The absolute quantification and percentage of complete vRNA in Lenti/βAS3-FB viral particles measured by ddPCR (n=9 dishes of identical cultures from three independent experiments; bars represent mean with SD; unpaired t test, *p<0.05, **p<0.01, ***p<0.001, ****p<0.0001). Panel D) Concentrations of physical particles in Lenti/βAS3-FB unconcentrated virus quantified by p24 ELISA (n=9 dishes of identical cultures from three independent experiments; bars represent mean with SD; unpaired t-test, *p<0.05, **p<0.01, ***p<0.001, ****p<0.0001). Panel E) Titers of Lenti/βAS3-FB, PYC-CAR, EFS-ADA, and Mini-G packaged in parental 293T and OAS1, PKR, and LDLR KO cells (n=9 dishes of identical cultures from three independent experiments; bars represent mean with SD; unpaired t test, *p<0.05, **p<0.01, ***p<0.001, ****p<0.0001).

FIG. 5, panels A-D, shows that packaging with transcription elongation factors SPT4/5 increased vRNA completeness and vector titer. Panel A) The fold difference of initial, intermediate, and complete vRNA compared with the transfection control and (panel B) a percentage of intermediate and complete vRNA in parental HEK293T cells with SPT4/5 plasmids or a filler plasmid (n=9 dishes identical cultures from three independent experiments; bars represent mean with SD; unpaired t test, *p<0.05, **p<0.01, ***p<0.001, ****p<0.0001). The percentage of intermediate vRNA was calculated as the copies of intermediate vRNA divided by the copies of initial RNA. The percentage of complete vRNA was calculated as the copies of complete vRNA divided by the copies of initial vRNA. Panel C) Titers of Lenti/βAS3-FB packaged in parental HEK293T cells with SPT4/5 plasmids or a filler plasmid as the transfection control (an unpackageable GFP plasmid without lentiviral sequences) (n=9 dishes identical cultures from three independent experiments; bars represent mean with SD; unpaired t-test, *p<0.05, **p<0.01, ***p<0.001, ****p<0.0001). Panel D) Concentration of physical particles measured by p24 ELISA (n=9-12 dishes with identical cultures from three to four independent experiments; bars represent mean with SD; unpaired t test, *p<0.05, **p<0.01, ***p<0.001, ****p<0.0001).

FIG. 6, panels A-D, shows that packaging with SPT4 and SPT5 in the CHEDAR cell line increased titer, vRNA, and physical particles. Panel A) Titers of Lenti/βAS3-FB packaged in parental HEK293T cells and CHEDAR cells with SPT4/5 plasmids or a filler plasmid as the transfection control (an unpackageable GFP plasmid without lentiviral sequences) (n=9 dishes identical cultures from three independent experiments; bars represent mean with SD; unpaired t test, *p<0.05, **p<0.01, ***p<0.001, ****p<0.0001). Panel B) The absolute quantification of complete vRNA and (panel C) the percentage of complete vRNA of Lenti/βAS3-FB packaged in parental HEK293T cells and CHEDAR cells with SPT4/5 plasmids or a filler plasmid as the transfection control. Panel D) The concentration of physical particles measured by p24 ELISA (n=6-9 dishes identical cultures from two to three independent experiments; bars represent mean with SD; unpaired t-test, *p<0.05, **p<0.01, ***p<0.001, ****p<0.0001).

FIG. 7, panels A-B, illustrates targeted restriction factor knockout screen in HEK 293T cells. Panel A) The workflow of the CRISPR-Cas9 screen. Single guide RNAs (sgRNA) were designed using the Benchling CRISPR online tool, and oligonucleotides were synthesized by Integrated DNA Technologies (San Diego, CA). The oligos were in vitro transcribed into the RNA form. Cas9 and sgRNA were delivered to 293T cells as RNP via electroporation. A portion of the edited cells was pelleted for gDNA extraction, PCR, Sanger sequencing, and ICE analysis for the KO score 24-48 hours after electroporation. The edited cells with greater than 20% indel were single-cell sorted. After two weeks of clonal expansion, the KO clones were selected by Sanger sequencing and then used for packaging to test whether they increased the titer of Lenti//βAS3-FB. If the KO clones successfully increased titer, the gene disruption was further validated on the protein level by western blot or flow cytometry. Panel B) Titers of Lenti//βAS3-FB in KO or KD clones (n=3-22 dishes of identical cultures from 1-6 independent experiments; bars represent mean with SD).

FIG. 8 shows maps of lentiviral vector proviruses. Long terminal repeats (LTR) include the ΔU3, R, U5 sequences. FB, FII-BEAD insulator. 3′ UTR, ß-globin gene 3′ UTR enhancer elements. P, promoter. HS2, HS3, HS4, ß-globin locus hypersensitive sites 2, 3, and 4. EC2, EC3, EC4, enhancer core elements. WPRE, mutated woodchuck hepatitis virus post-transcriptional regulatory element. β-Globin, β-Globin gene cassette. EFS, elongation factor 1 alpha promoter. ADA, codon-optimized adenosine deaminase cDNA. PYC-CAR encodes the CD8 signal peptide, VL, linker, VH, CD8-alpha hinge and transmembrane (TM), CD137 (41BB) co-stimulatory cytoplasmic signaling domain, and CD3-zeta cytoplasmic signaling domain expressed under the control of the MND promoter.

FIG. 9, panels A-D, shows that restoring protein expressions of IFNAR1 and ATR did not decrease titer. Panel A) ATR protein expression in parental HEK293T, ATR KD 293T, ATR KD 293T transduced with Lenti/GFP, and ATR KD 293T transduced with Lenti/ATR measured by western blot. R-actin was used as a loading control. ATR KD cells were transduced with an LV encoding MND-ATR-ires-GFP or an LV encoding MND-GFP as a transduction control. Two days after transduction, the transduced cells were sorted for the GFP+ population. The GFP+ cells were rested for an additional 12 days, measured for VCN analysis by ddPCR, and used for packaging of Lenti/βAS3-FB. Panel B) Titers of Lenti/βAS3-FB packaged in parental HEK293T, ATR KD 293T, ATR KD 293T transduced with Lenti/GFP, and ATR KD 293T transduced with Lenti/ATR. (n=9 dishes of identical cultures from 3 independent experiments; bars represent mean with SD; one-way ANOVA; ns, not significant). Panel C) Protein expression of IFNAR1 in parental 293T, IFNAR1−/− 293T, IFNAR1−/− 293T transduced with Lenti/GFP, and IFNAR1−/− 293T transduced with Lenti/IFNAR1 measured by western blot. β-Actin was used as a loading control. Panel D) Titers of Lenti/βAS3-FB packaged in parental 293T, IFNAR1−/− 293T, IFNAR1−/− 293T transduced with Lenti/GFP, and IFNAR1−/− 293T transduced with Lenti/IFNAR1 (n=9 dishes of identical cultures from 3 independent experiments; bars represent mean with SD; one-way ANOVA; ns, not significant).

FIG. 10 shows that additional KO of IFNAR1 and ATR did not increase titer. To create double KO, Cas9 and sgRNA targeting OAS1 were electroporated into the PKR−/− isogenic clone. After confirming the cutting efficiency, some of the edited cells were sorted for single-cell clones, and the other cells were electroporated with Cas9 and sgRNA targeting LDLR. This process was repeated to further knock out IFNAR1 and at the end knock down ATR. Electroporation of two sgRNA simultaneously was avoided to prevent chromosome translocation. The sgRNA were introduced in the following order—PKR, OAS1, LDLR, IFNAR1, and ATR—to generate single, double, triple, quadruple, and quintuple KO/KD cells. All the edited cells were sorted for single-cell clones. The clones with no parental allele or 30% parental allele for ATR were expanded for packaging of Lenti//βAS3-FB. OPL (subsequently renamed as CHEDAR), OAS1 PKR LDLR. OPLI, OAS1, PKR, LDLR, IFNAR1. OPLIA, OAS, PKR, LDLR, IFNAR1, ATR. The numbers after the names are the clone numbers.

FIG. 11, panels A and B, illustrates the morphology and growth rate of CHEDAR. Panel A) The morphology of parental HEK293T cells and the CHEDAR cell line at 40×. Panel B) The growth rate of parental HEK293T cells and the CHEDAR cells (OAS1, PKR, LDLR triple KO). The cells were plated at equal densities, and the cell numbers were monitored every 24 hours for three days (n=3 dishes of identical cultures from one independent experiment).

### DETAILED DESCRIPTION

Retroviral vectors are created by removal of one or more of the retroviral gag, pol, and env genes. These are then replaced by a transgene (e.g., a therapeutic gene) that is to be expressed by the retroviral vector. In order to produce vector particles a packaging cell is essential. Packaging cell lines typically provide all the viral proteins required for capsid production and the virion maturation of the vector. Typically, these packaging cell lines have been made so that they contain the gag, pol and env genes. Following insertion of the desired gene into in the retroviral DNA vector, and introduction of the retroviral DNA vector into a packaging cell line, it relatively straightforward now simple matter to prepare retroviral vectors.

However, it has been observed that titers of retroviral vectors (e.g., lentiviral vectors (LVs)) decrease substantially with increasing proviral length. The reduction in titer, especially in the vectors with complex expression cassettes, creates a barrier for scaling up vector production for clinical use. To address this issue, improved retroviral packaging cells are provided that produce increased viral titer.

In particular, as described in the Examples herein, a targeted CRISPR-mediated knockout screen was conducted in HEK293T cells to identify host cell factors that restrict virus production. As described the examples, knockout of one or more genes that regulate the DNA damage response pathway, transcription, and innate immunity was investigated. It was shown that knockout of one or more genes selected from the group consisting of OAS1, ATR, IFNAR1, and/or PKR increased the titer of a packaged lentiviral vector (B-globin LV for the treatment of sickle cell disease).

Similarly, knockout of one or more genes selected from the group consisting of IFNAR1, ATR, OAS1, LDLR increased viral titer. Additionally, knocking out PKR, OAS1, and LDLR in 293T cells additively increased titer and RNA production.

Accordingly, in certain embodiments, packaging cells are provided comprising a knockout of one or more mammalian genes whose expression results in lower viral titers when transfected with a defective recombinant retroviral genome are knocked out. In certain embodiments the packaging cell comprises a knockout of one or more mammalian genes that regulate the DNA damage response pathway, and/or that regulate transcription, and/or that regulate innate immunity. In certain embodiments the packaging cell comprises a knockout of one or more genes selected from the group consisting of ATR (a gene that encodes serine/threonine-protein kinase ATR also known as ataxia telangiectasia and Rad3-related protein or FRAP-related protein 1), IFNAR1 (Interferon Alpha and Beta Receptor Subunit 1), IFNAR2 (Interferon Alpha and Beta Receptor Subunit 2), LDLR (low density lipoprotein receptor gene), PKR (a gene that encodes protein kinase R), and a member of the 2′-5′-oligoadenylate synthetase gene family. While data are illustrated for the OAS1 gene, it is noted that the OAS gene family includes, inter alia, OAS1, OAS2, OAS3, and OASL and in various embodiments, knockout of any one or more of these OAS genes is contemplated.

In certain embodiments the packaging cells described herein comprise a knockout of one or more of an OAS gene family member (e.g., OAS1), ATR, IFNAR, and PKR. In certain embodiments packaging cells described herein comprise a knockout or knockdown of all of an OAS gene (e.g., OAS1), ATR, IFNAR, and PKR. In certain embodiments packaging cells described herein comprise a knockout or knockdown of one or more of IFNAR1, ATR, OAS1, and LDLR. In certain embodiments, packaging cells described herein comprise a knockout or knockdown of all of IFNAR1, ATR, OAS1, and LDLR. In certain embodiments packaging cells described herein comprise a knockout or knockdown of one or more of LDLR and OAS1. In certain embodiments packaging cells described herein comprise a knockout or knockdown of both LDLR and OAS1. In certain embodiments packaging cells described herein comprise a knockout or knockdown of one or more of PKR, OAS1, and LDLR. In certain embodiments packaging cells described herein comprise a knockout of all of PKR, OAS1, and LDLR.

In certain embodiments the packaging cells described herein comprises a knockout or knockdown of one or more genes selected from the group consisting of 2′-5′-oligoadenylate synthetase 1 (OAS1), low-density lipoprotein receptor (LDLR), and PKR. In certain embodiments the packaging cells comprise a knockout or knockdown of OAS1 and LDLR, or OAS1 and PKR, or LDLR and PKR. In certain embodiments the packaging cells comprise a knockout or knockdown of OAS1, LDLR, and PKR.

It was also observed that expressing transcription elongation factors SPT4 and/or SPT5 improved RNA production, thereby increasing titer. Accordingly, in certain embodiments, the packaging cell line is modified to express transcription elongation factor SPT4/SPT5. In certain embodiments constructs comprising expression cassettes for the SPT4/SPT5 complex are introduced into the packaging cell using methods well known to those of skill in the art. In certain embodiments the SPT4/SPT5 expression cassettes remain episomal within the packaging cell, while in other embodiments the SPT4/SPT5 expression cassettes are designed to integrate (e.g., stably integrate) into the packaging cell genome. In certain embodiments expression of the SPT4/SPT5 complex is under the control of a constitutive promoter, while in other embodiments, expression of the SPT4/SPT5 complex is under the control of an inducible promoter.

By using packaging cell lines described herein, it is possible to propagate and isolate quantities of retroviral vector particles (e.g., to prepare suitable titers of the retroviral vector particles) for subsequent use (e.g., gene therapy) and it is believed the packaging cells comprising one or more knockouts as described herein provide viral titers that are significantly elevated compared to those produced by packaging cells without the knockout.

It is noted that different packaging cells lines can provide different envelope protein(s) (e.g., ecotropic, amphotropic or xenotropic) to be incorporated into the viral capsid. In certain embodiments, this envelope protein determining the specificity of the viral particle for particular target cells (e.g., ecotropic for murine and rat; amphotropic for most mammalian cell types including human, dog and mouse; and xenotropic for most mammalian cell types except murine cells). The appropriate packaging cell line may be used to ensure that particular target cells are targeted by the packaged viral particles. Methods of introducing the retroviral vectors comprising the nucleic acid encoding the reprogramming factors into packaging cell lines, and of collecting the viral particles that are generated by the packaging lines are well known in the art.

An illustrative packaging cell contains genes encoding Gag and Pol, as well as the desired envelope protein, but does not contain the packaging signal “psi” or the viral LTRs. Thus, a packaging cell can only form empty virion particles. However, once a retroviral RNA genome (which contains the nucleotide sequence of interest), but not genes for env, gag, or pol is introduced into the packaging cell, the packaging cell can produce retroviral particles that in some embodiments may be pseudotyped. Packaging cells thus provide the missing retroviral components (i.e., the components for which the retroviral genome is defective) essential for viral replication in trans.

Methods for production of replication-deficient retroviral genomes containing a nucleotide sequence of interest, as well as methods for generating a cell line expressing the env, gag and pol genes, are well known in the art and are described in, for example, U.S. Pat. No. 4,861,719; PCT Published Application No. WO 92/005266; PCT Published Application No. WO 92/014829, and the like, each of which are incorporated herein by reference with respect to production of replication-deficient retroviral genomes and packaging cell lines expressing retroviral gag and pol genes.

Retroviral packaging cell lines can be derived from any mammalian or non-mammalian cell that can express the retroviral Gag and Pol proteins and can express the desired envelope protein. In certain embodiments illustrative, but non-limiting embodiments the cell line from which the packaging cell line is derived is a cell selected from a liver, stroma, myogenic, fibroblast, and embryonic stem cell. Illustrative cells used by those of skill in the art for development of packaging cell lines include: 293 (ATCC CCL X) HeLa (ATCC CCL 2), D17 (ATCC CCL 183), MDCK (ATCC CCL 34), BHK (ATCC CCL-10), or Cf2Th (ATCC CRL 1430) cell, most preferably a 293 cell, each of which are publicly available from the ATCC. In certain embodiments the packaging cell comprises a may be a human cell line, such as for example HEK293, HEK293T, TE671, HT1080, 3T3, K562, 3T3, U937, H9, and the like.

Methods of generating packaging cells are well known to those of skill in the art. In one illustrative, but non-limiting approach, the nucleic acid sequences encoding retroviral Gag, Pol and Env proteins are introduced into the cell and stably integrated into the cell genome to produce a stable packaging cell line. As used herein, the term “stably integrated” means that the foreign genes (e.g., Gag, Pol, and/or Env) become integrated into the cell's genome. This packaging cell line produces the proteins required for packaging retroviral RNA but it cannot bring about encapsidation, e.g., due to the lack of a psi region. However, when a defective retrovirus nucleic acid transgene construct (having a psi region) is introduced into the packaging cell line, the helper proteins can package the psi-positive retrovirus nucleic acid transgene construct to produce the recombinant virus stock.

A second illustrative, but non-limiting approach is to introduce the different DNA sequences that are required to produce a retroviral vector particle (e.g., the env coding sequences, the gag-pol coding sequence and the defective retroviral genome containing a transgene of interest (i.e. the retrovirus nucleic acid transgene construct) into the cell at the same time by transient transfection and the procedure is referred to as transient triple transfection. PCT Publication WO 94/029438 describes the production of packaging cells in vitro using this multiple DNA transient transfection method.

It will be recognized that in certain embodiments, the packaging cells described herein do not contain the gag, env, or pol genes, but comprise the gene knockouts described above. Such packaging cells will be suitable for retroviral production using transient transfection methods, e.g., as described above. In certain embodiments the packaging cells comprise the viral sequences necessary for packaging either as episomal constructs or stably integrated into the packaging cell genome, in which case the packaging cells only need to be transfected with a defective, recombinant retroviral genome to produce the desired viral vector.

As noted above, the cells used as packaging cells described herein comprise one or more knockouts of mammalian genes. In certain embodiments the cells comprise one or more knockout or knockdown genes selected from the group consisting of ATR, IFNAR1, LDLR, an OAS gene (e.g., OAS1), and PKR.

Numerous methods to knock out genes in mammalian cells are well known to those of skill in the art. Traditionally, homologous recombination was the main method for producing a gene knockout. This method involves creating a DNA construct containing the desired mutation. For knockout purposes, this typically involves a drug resistance marker in place of the desired knockout gene. In various embodiments the construct will also contain a region homologous (e.g., often a minimum of 2 kb of homology) to the target sequence. The construct is then delivered to target cells either through microinjection, lipofection, electroporation, and the like. This method relies on the cell's own repair mechanisms to recombine the DNA construct into the existing DNA resulting in the sequence of the gene being altered, and most cases the gene is translated into a nonfunctional protein, if it is translated at all. However, homologous recombination was an inefficient process as homologous recombination typically accounts for only about 10⁻² to 10⁻³ of DNA integrations (see, e.g., Santiago et al. (2008) Proc. Natl. Acad. Sci. USA, 105(15): 5809-5814).

Accordingly, more efficient methods of generating gene knockouts utilize a site-specific nuclease to introduce the desired mutation into a specific target site in the cell genome. Suitable site-specific nucleases include, but are not limited to zinc-finger nucleases, transcription activator-like effector nucleases (TALENs), and CRISPR (Clustered regularly interspaced short palindromic repeats) Cas constructs. These site-specific nucleases all can be used to precisely target a DNA sequence in order to introduce a double-stranded break. Once this occurs, the cell's repair mechanisms attempt to repair this double stranded break, often through non-homologous end joining (NHEJ), which involves directly ligating the two cut ends together (Id.) This may be done imperfectly, therefore sometimes causing insertions or deletions of base pairs, which cause frameshift mutations. These mutations can render the gene in which they occur nonfunctional, thus creating a knockout of that gene. In certain embodiments the site-specific nuclease is delivered along with a nucleotide sequence designed to be inserted into the cut site and effectively disrupt expression of the gene and/or production of an active protein product. In any case the use of site-specific nucleases is more efficient and often more specific, than homologous recombination, and therefore can be more easily used to create knockouts (Id.). Thus, in certain embodiments, the gene knockouts described herein are produced by use of a zinc-finger nuclease, or a TALEN, and most preferably a CRISPR/Cas construct.

In this regard, it is noted that Zinc-finger nucleases consist of DNA binding domains that can precisely target a DNA sequence. Each zinc finger can recognize codons of a desired DNA sequence, and therefore can be modularly assembled to bind to a particular sequence. These binding domains are coupled with a restriction endonuclease that can cause a double stranded break (DSB) in the DNA.

TALENS (Transcription activator-like effector nucleases) also contain a DNA binding domain and a nuclease that can cleave DNA. The DNA binding region consists of amino acid repeats that each recognize a single base pair of the desired targeted DNA sequence. [5] If this cleavage is targeted to a gene coding region, and NHEJ-mediated repair introduces insertions and deletions, a frameshift mutation often results, thus disrupting function of the gene.

Clustered regularly interspaced short palindromic repeats (CRISPR)/Cas9 is a method for genome editing that contains a guide RNA complexed with a Cas9 protein. The guide RNA can be engineered to match a desired DNA sequence through simple complementary base pairing, as opposed to the time-consuming assembly of constructs required by zinc-fingers or TALENs. The coupled Cas9 will cause a double stranded break in the DNA. Following the same principle as zinc-fingers and TALENs, the attempts to repair these double stranded breaks often result in frameshift mutations that result in an nonfunctional gene.

While essentially any known method can be used to knockout the genes in the packaging cells described herein, Example 1 and 2 simply utilized CRISPR/Cas9 to produce the knockout(s). Illustrative guide RNAs that can readily be used to target the various genes described herein include but are not limited to the guide RNAs shown in Table 1.

TABLE 1
Illustrative single strand guide
RNAs used to knock out genes.

| Target | | SEQ ID |
|---|---|---|
| Gene | Guide RNA (sgRNA) | NO |
| IFNAR1 | AAACACTTCTTCATGGTATG | 1 |
| OAS1 | CTGAAGGAAAGGTGCTTCCG | 2 |
| ATR | AAAGTGCTAGCTGGTTGTGC | 3 |
| LDLR | GTCTGTCACCTGCAAATCCG | 4 |
| PKR | AATACATACCGTCAGAAGCA | 5 |

The foregoing packaging cells and methods are illustrative and non-limiting. Using the teaching provided herein numerous other packaging cells that produce high titers of virion will be available to one of skill in the art.

### Examples

The following examples are offered to illustrate, but not to limit the claimed invention.

### Example 1

### 293T Packaging Cells with Targeted Gene Knockouts that Improve Lentiviral Vector Titers

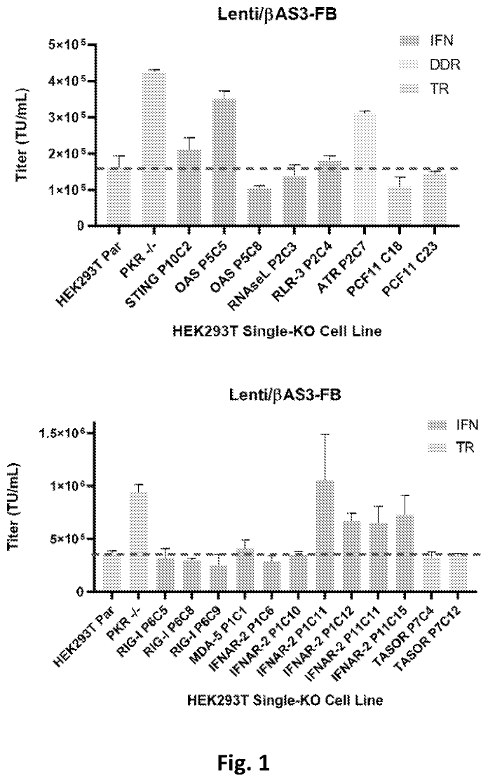

We conducted a targeted CRISPR-mediated knockout screen in HEK293T cells to identify host cells factors that restrict virus production. In particular, we designed guide RNAs to target various genes that regulate the DNA damage response pathway, transcription, and innate immunity. We identified four host cell antiviral factors that reduce titers: OAS1, ATR, IFNAR1, and PKR (see, e.g., FIG. 1). Knocking out these factors increased the titer of β-globin LV for the treatment of sickle cell disease by 2-4 fold.

Ongoing study as described in Example 2, below, test more antiviral factors and the combinatorial effects of multiple knockouts. In certain embodiments an ultimate packaging cell line is developed with many antiviral factors removed to increase titer and infectivity of the LVs produced. The increase in titer and gene transfer should support more effective gene therapy for the hemoglobinopathies and other conditions where large transgenes are needed.

### Example 2

### Improved Lentiviral Vector Titers from a Multi-Gene Knockout Packaging Line

Lentiviral vectors (LVs) are robust delivery vehicles for gene therapy as they can efficiently integrate transgenes into host cell genomes. However, LVs with lengthy or complex expression cassettes typically are produced at low titers and have reduced gene transfer capacity, creating barriers for clinical and commercial applications. Modifications of the packaging cell line and methods may be able to produce complex vectors at higher titer and infectivity and may improve production of many different LVs. In this example, we identified two host restriction factors in HEK293T packaging cells that impeded LV production, 20-50-oligoadenylate synthetase 1 (OAS1) and low-density lipoprotein receptor (LDLR). Knocking out these two genes separately led to ˜2-fold increases in viral titer. We created a monoclonal cell line, CRISPRed HEK293T to Disrupt Antiviral Response (CHEDAR), by successively knocking out OAS1, LDLR, and PKR, a previously identified factor impeding LV titers. Packaging in CHEDAR yielded ˜7-fold increases in physical particles, full-length vector RNA, and vector titers. In addition, overexpressing transcription elongation factors, SPT4 and SPT5, during packaging improved the production of full-length vector RNA, thereby increasing titers by ˜2-fold. Packaging in CHEDAR with over-expression of SPT4 and SPT5 led to ˜11-fold increases of titers. These approaches improved the production of a variety of LVs, especially vectors with low titers or with internal promoters in the reverse orientation, and are believed to be beneficial for multiple gene therapy applications.

### Introduction to Example 2

Transplantation of autologous hematopoietic stem cells modified by lentiviral-based gene therapy has successfully treated multiple genetic blood cell diseases.¹⁻⁴ Lentiviral vectors (LVs) allow integration of transgenes into the host cell genomes of both dividing and nondividing cells, providing long-term stable expression of the gene of interest. However, we and others have observed that LV titers and infectivity decrease with increasing proviral length, resulting in less efficient transduction of patient cells as well as increased costs for clinical and commercial applications. Optimizing the expression cassette is one strategy to create LVs with optimal titer, infectivity, and expression^{5,6} but may be a prolonged process and have to be applied to each individual LV. Improvements of the vector packaging platform or the manufacturing protocol can provide a global solution to the production of many different LVs.

Recent research has shown that many cellular restriction factors (RFs) inhibit specific steps of the lentiviral life cycle.⁷⁻⁹ Some RFs are inducible by sensing viral components and activating the interferon (IFN) signaling cascade, while other RFs are ubiquitously expressed for direct antiviral functions. Ferreira et al. reported that LV production titer is not limited by induced intracellular innate immunity in HEK293T cells, because there was no detectable IFN cytokine release during the packaging process.¹⁰ This is likely due to the large T-antigen (TAg) and adenovirus E1A, which are expressed in HEK293T and inactivate the tumor suppressors p53, IRF3, and other IFN-dependent transcription downstream of RNA and DNA sensing.¹¹⁻¹⁴

On the other hand, constitutively expressed antiviral effectors appeared to regulate vector production in HEK293T cells. An example is protein kinase R (PKR), an IFN-stimulated gene that regulates protein synthesis. PKR is constitutively expressed in all tissues in an inactive form and is upregulated by type I and type III IFNs.¹⁵ PKR can be activated by TAR sequence or double-stranded RNA to inhibit general translation and hence viral protein production.^{16,17} Knocking out PKR in HEK293T cells increased titers of LVs, particularly for vectors with internal promoters in the reverse orientation, a common configuration for b-globin expressing LVs.^{18,19} Based on these previous studies, it is conceivable that the constitutively expressed antiviral effectors can still restrict LV production in HEK293T cells.

Moreover, RFs are not limited only to antiviral effectors but also include genes that regulate the lentiviral life cycle. To date, the most common envelope glycoprotein used to pseudotype LVs is the vesicular stomatitis virus spike protein G (VSVG), due to its robust and pantropic infectivity. The low-density lipoprotein receptor (LDLR) serves as the major entry port for VSVG-pseudotyped LVs, while other LDLR family members serve as alternative receptors.^{20,21} Otahal et al. showed that LDLR prematurely interacts with VSVG in an ER-Golgi intermediate compartment and reroutes the LDLR-VSVG complex to aggresome/autophagosome degradation prior to particle release.²² Therefore, LDLR is likely to be an RF that regulates the levels of VSVG that are available for vector pseudotyping. The effects on vector production from RFs that regulate antiviral responses or interfere in the lentiviral life cycle remain to be fully explored.

Another impediment to efficient vector production is the truncation of vRNA (vRNA). Previously we reported that vRNAs were truncated in a vector length-dependent manner in packaging cells and these truncated genomes were exported into the viral particles.¹⁹ These truncated vRNAs failed reverse transcription at the first strand transfer step due to the absence of the 30 long terminal repeat (LTR) and could not form the double-stranded viral DNA to be integrated into the host cell genome. Expression during packaging of Tat, an HIV-1 accessory protein that functions to increase transcriptional processivity, modestly increased titers and the levels of complete vRNA, but the percentage of complete vRNA did not increase. New strategies are needed to improve genomic RNA completeness for more efficient complex LVs. As described in this example, we conducted a targeted CRISPR-Cas9-mediated knockout (KO) screen in HEK293T cells to disrupt RFs that inhibit specific steps of the lentiviral life cycle. We showed that knocking out PKR, OAS1, and LDLR additively increased the titer of various LVs by ˜7-fold, particularly complex and lengthy LVs with low titers. We named the triple-KO cell line CRISPRed HEK293T to Disrupt Antiviral Response (CHEDAR). In addition, overexpressing transcription elongation factors SPT4 and SPT5 improved vRNA completeness, thereby increasing the titer of LVs by ˜2-fold. Packaging with the transcription elongation factors in CHEDAR cell line increased titer of the β-globin vector by ˜11-fold. These modifications of the packaging cell line and protocol should support the applications of different LVs in gene and cell therapy by improving the yield of vector production.

### Results.

Knocking out IFNAR1, ATR, OAS1, and LDLR in 293T cells increased titers

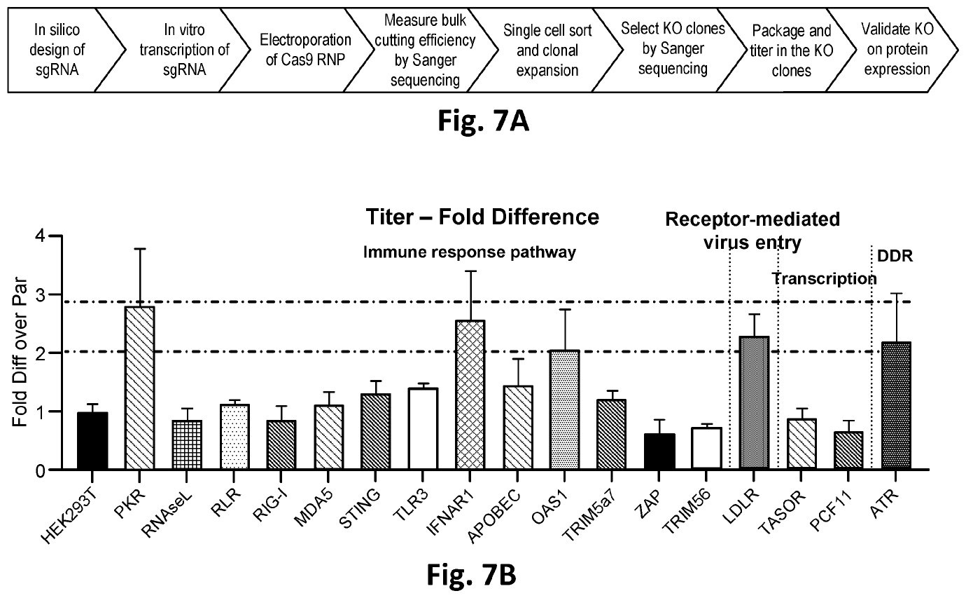

To identify RFs that limit LV production, we conducted a targeted CRISPR-Cas9 KO screen in HEK293T cells with a focus on 16 genes, each of which regulates one of the following biological properties: the immune response, DNA damage response, receptor-mediated virus entry, and transcription. The Cas9 and single guide RNAs (sgRNAs) were delivered to HEK293T cells as ribonucleoprotein (RNP) via electroporation, and the edited cells were sorted for single-cell clones (FIG. 7, panel A). KO or knockdown (KD) clones were validated on the genomic level by Inference of CRISPR Edits (ICE) analysis and on the protein level by western blot or flow cytometry. The sequences of the sgRNAs used in this study and the genomic profile of the clones are listed in Table 2 and Table 3.

TABLE 2
Genomic profile of KO cells assessed
by ICE Synthego.

| | | | Knock- | | SEQ |
|---|---|---|---|---|---|
| | sgRNA | Indel | out | Indel | ID |
| Gene | Sequence | % | Score | Pattern | NO |
| IFNAR1 | AAACACTTCT | 99 | 99 | 99% +1 | 1 |
| | TCATGGTATG | | | | |
| OAS1 | CTGAAGGAAA | 96 | 96 | 49% +2 | 2 |
| | GGTGCTTCCG | | | 47% +1 | |
| ATR | AAAGTGCTAG | 63 | 63 | 35% −2 | 3 |
| | CTGGTTGTGC | | | 35% 0 | |
| | | | | 28% −13 | |
| LDLR | GTCTGTCACC | 99 | 99 | 99% +1 | 4 |
| | TGCAAATCCG | | | | |
| PKR | AATACATACC | 93 | 51 | 51% −41 | 5 |
| | GTCAGAAGCA | | | 37% −6 | |

Isogenic gene edited cells were harvested for genomic DNA extraction. The sgRNA targeted regions were PCR amplified and sent for Sanger sequencing. The sequencing results were then analyzed using ICE Synthego for cutting efficiencies and indel patterns.

TABLE 3
sgRNA sequences and genomic profile of KO
cells assessed by ICE Synthego analysis.
Isogenic cells were harvested for genomic
DNA extraction. The sgRNA targeted region
was PCR amplified and sent for Sanger
sequencing. The sequencing results were
then analyzed using ICE Synthego.

| | | | Knockout | | |
|---|---|---|---|---|---|
| | | | score | | SEQ |
| | sgRNA | Indel | Indel | | ID |
| Gene | sequence | % | Pattern | | NO |
| STING | CGGGCCGACC | 96 | 96 | 39% −20 | 6 |
| | GCATTTGGGA | | | 21% −11 | |
| | | | | 16% −8 | |
| | | | | 8% −11 | |
| | | | | 6% −8 | |
| | | | | 4% −11 | |
| | | | | 2% −8 | |
| RNAseL | TTATCCTCGC | 95 | 76 | 52% +1 | 7 |
| | AGCGATTGCG | | | 20% −6 | |
| | | | | 23% −1 | |
| RLR3 | TGGACCGTGA | 97 | 97 | 32% +1 | 8 |
| | CAACCCTGAG | | | 29% −4 | |
| | | | | 22% −20 | |
| | | | | 6% −4 | |
| | | | | 5% −20 | |
| | | | | 3% −20 | |
| RIG-I | AAACAACAAG | 99 | 99 | 99% +1 | 9 |
| | GGCCCAATGG | | | | |
| MDA5 | CGAATTCCCG | 100 | 100 | 100% −4 | 10 |
| | AGTCCAACCA | | | | |
| TASOR | GATAGGCAAA | 98 | 98 | 98% +1 | 11 |
| | AAAGCACGAG | | | | |
| PCF11 | TCTGCTCTGA | 57 | 57 | 57% −8 | 12 |
| | CATTGCGCCG | | | | |
| TLR3 | GTACCTGAGT | 95 | 95 | 37% −20 | 13 |
| | CAACTTCAGG | | | 23% −11 | |
| | | | | 16% −11 | |
| | | | | 7% −11 | |
| | | | | 7% −20 | |
| | | | | 3% −20 | |
| | | | | 2% −11 | |
| TRIP12 | GGTCACTGCG | 98 | 98 | 98% +1 | 14 |
| | ACGTTCACAG | | | | |
| TRIM5a | GATCTGAGAT | 100 | 100 | 71% −4 | 15 |
| | GAGCTCTCTC | | | 29% −4 | |
| TRIM56 | GAAGAAGTTG | 95 | 95 | 47% +1 | 16 |
| | GTCTTGAAGG | | | 30% −11 | |
| | | | | 18% −11 | |
| ZAP | GCAACTATTC | 97 | 97 | 33% −2 | 17 |
| | GCAGTCCGAG | | | 32% −4 | |
| | | | | 32% −13 | |
| APOBEC | CTGTCCTAAA | 94 | 94 | 73% −13 | 18 |
| 3G | ACCAGAAGCT | | | 21% −14 | |

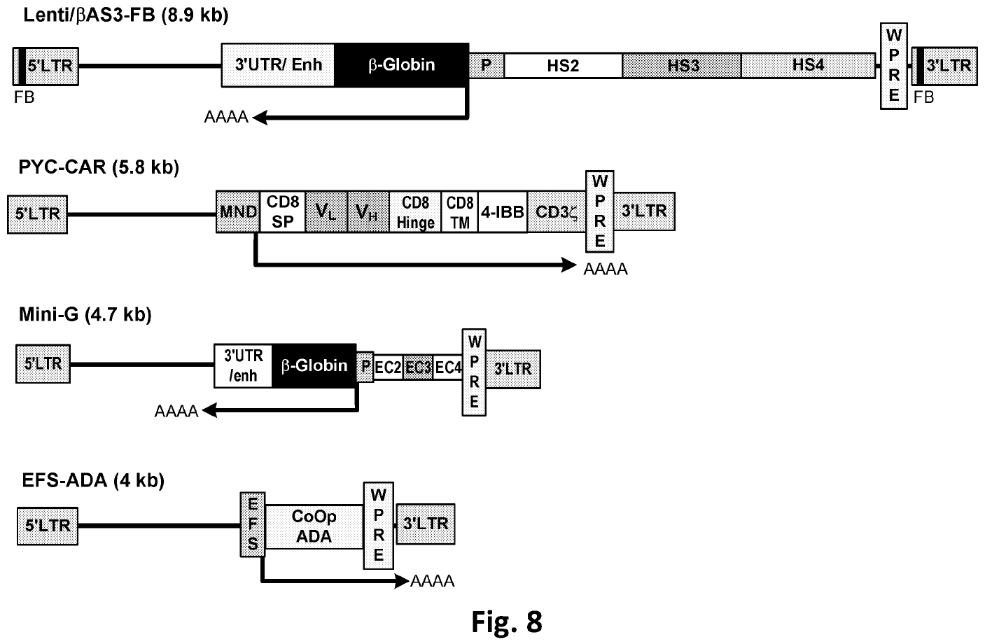

These single-cell clones were expanded to test whether they increased the titer of the Lenti/βAS3-FB LV, which is known to have low titer and infectivity. Previously described by Romero et al. and Han et al.,^{19,23} Lenti/βAS3-FB is an 8.9-kb LV carrying a complex anti-sickling β-globin gene cassette in reverse orientation, as shown in FIG. 8. PKR−/− cells were used as a positive control for the packaging process because we previously showed that knocking out PKR increased the titers of reverse-oriented LVs, such as Lenti/βAS3-FB.²⁴

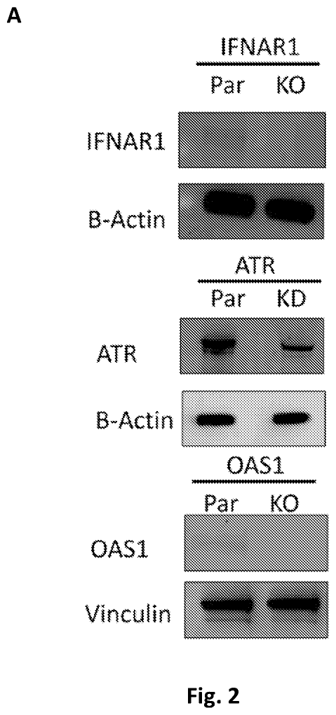

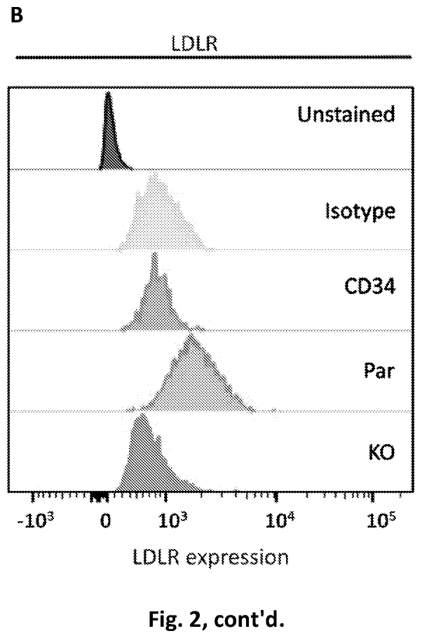

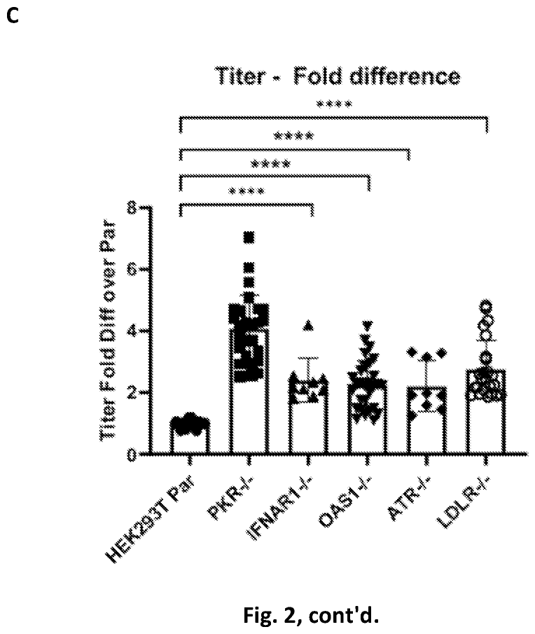

Because homozygous deletion of ATR leads to chromosome breaks and proliferative failure in culture,^{25,27} we selected an ATR KD clone with 63% indel and 63% KO score that showed a significant reduction in protein expression (Table 2 and FIG. 2, panel A). Interferon Alpha and beta receptor subunit 1 (IFNAR1), OAS1, and LDLR were successfully knocked out, as shown in FIG. 2, panels A and B. Unstimulated CD34+ hematopoietic stem and progenitor cells (HSPCs) are known to have no or low expression of LDLR²⁸ and therefore were used as a negative control when measuring LDLR expression in the LDLR KO cells by flow cytometry (FIG. 2, panel B).

The targeted CRISPR-Cas9 screen revealed that disruption individually of IFNAR1, OAS1, ATR, and LDLR genes each increased titers by ˜2-fold (FIG. 2, panel C; n=9-29 dishes of identical cultures from three to 10 independent experiments; bars represent mean with standard deviation (SD); unpaired t test, *p<0.05, **p<0.01, ***p<0.001, ****p<0.0001). The PKR−/− cells increased titer by 4.1-±1.1-fold. The IFNAR1−/− cells increased titer by 2.4-±0.7-fold. The OAS1−/− cells increased titer by 2.3-±0.8-fold. The ATR knockdown cells increased titer by 2.2-±0.8-fold. The LDLR−/− cells increased titer by 2.8-±0.9-fold. Knocking out RLR, RIG-I, MDA5, STING, TLR3, APOBEC, TRIM5a, TRIM56, ZAP, TASOR, and PCF11 in HEK293T cells did not increase the titer of Lenti/βAS3-FB more than 2-fold (FIG. 7, panel B; n=3-22 dishes of identical cultures from one to six independent experiments; bars represent mean with SD). A potential explanation is that HEK293T cells do not have intact cellular pathways for the actions of these genes. Therefore, we decided to not prioritize these targets for this study.

Decreased Titers are Associated with Rescued LDLR and OAS1 Expression

To validate the RFs that increased titer, we restored the protein expression of the four RFs in their respective KO clones. The wildtype (WT) cDNAs of the longest open reading frames were cloned into a lentiviral backbone with an IRES-GFP reporter gene driven by the MND U3 promoter. The KO cells were transduced with a lentivirus encoding the WT cDNA linked to ires-GFP or a lentivirus encoding MND-GFP as a transduction control. The cells were subsequently sorted for the GFP+ population to exclude the non-transduced cells.

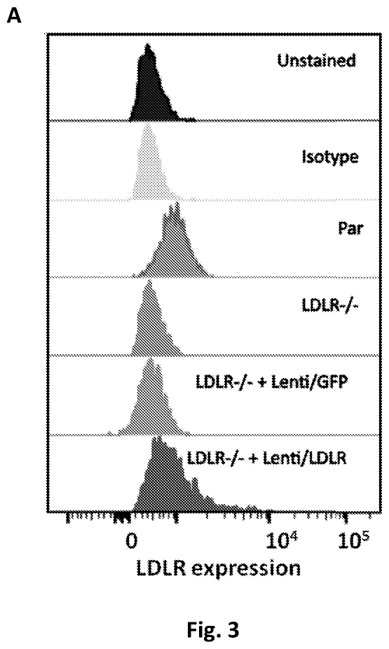

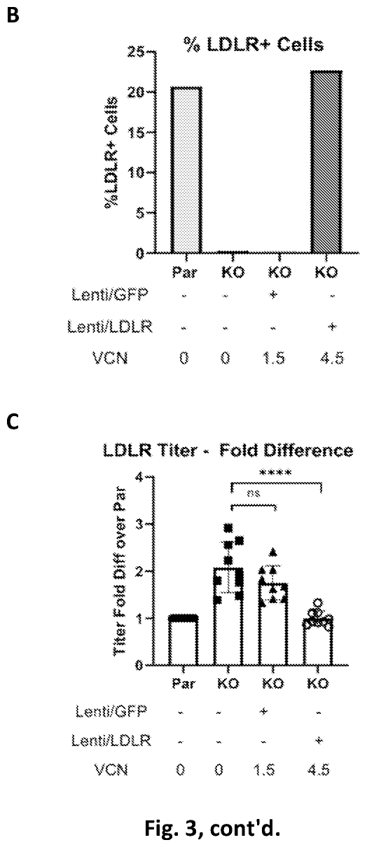

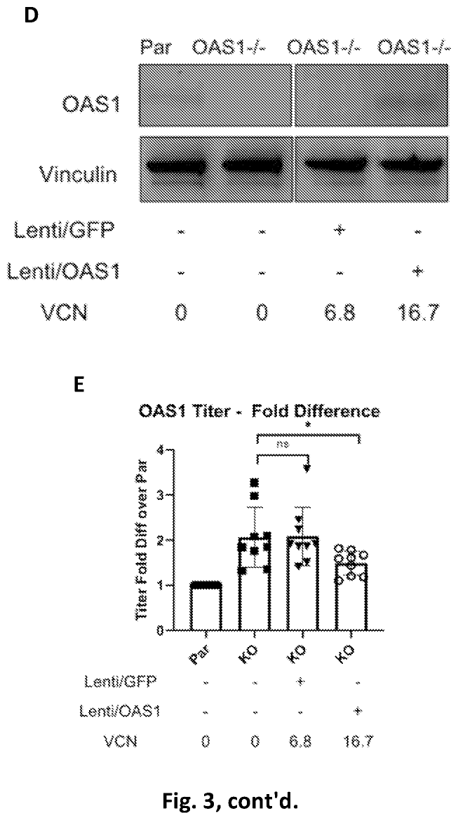

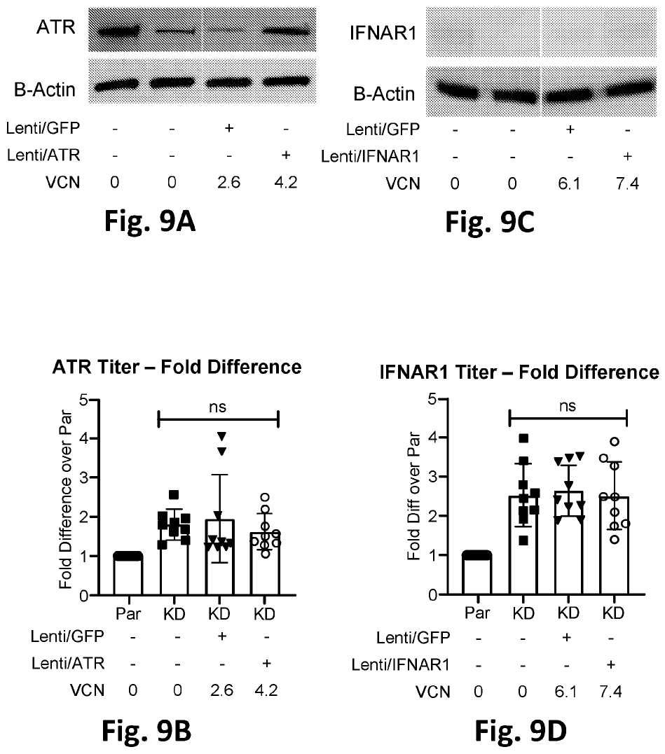

We first confirmed the restoration of the protein expressions of LDLR, OAS1, ATR, and IFNAR1 in the KO/KD cells by western blot or flow cytometry (FIGS. 3, panels A, B, and D, FIG. 9, panels A and C). The cells transduced with lentivirus encoding the WT cDNAs showed protein expression levels that were comparable with expression in the parental HEK293T cells, while the cells transduced with the control MND-GFP LV did not show restoration of the target proteins.

Next, we packaged Lenti/βAS3-FB in parental HEK293T, KO cells, KO cells transduced with an LV encoding the corresponding cDNA, or KO cells transduced with control MND-GFP LV. Restoring LDLR expression decreased the titer to the WT level, while the LDLR−/− cells transduced only with MND-GFP showed persistence of the increased titer (n=9 dishes of identical cultures from three independent experiments; bars represent mean with SD; unpaired t test, *p<0.05, **p<0.01, ***p<0.001, ****p<0.0001). Restoring OAS1 expression decreased titer compared with the OAS1−/− cells and OAS1−/− cells transduced with MND-GFP (n=12 dishes of identical cultures from four independent experiments; bars represent mean with SD; unpaired t test, *p<0.05, **p<0.01, ***p<0.001, ****p<0.0001). The decrease in titer in the cells in which LDLR and OAS1 were restored suggested that the titer changes were associated with these genes. Although we observed titer increase in multiple IFNAR1−/− clones (data not shown), restoring IFNAR1 and ATR did not decrease titers (FIGS. 9, panels B and D; n=8 or 9 dishes of identical cultures from three independent experiments; bars represent mean with SD; unpaired t test; ns, not significant). The failure to restore decreased titers could be due to the introduction of an isoform of the protein that was not responsible for the antiviral activity observed upon gene disruption.

### Combining OAS1, LDLR, and PKR Gene Knockouts LED to Increased Physical Particle Formation, RNA Production, and Titers

Because OAS1, LDLR, and PKR are likely to have nonredundant functions, it is conceivable that knocking out multiple RFs may additively improve the vector production process. Next, we consecutively knocked out these RFs to study the effects on titer. Cas9 and sgRNA targeting OAS1 were electroporated into the PKR−/− isogenic clone. After confirming the cutting efficiency at OAS1, some of the edited cells were sorted for single-cell clones; other cells were again electroporated with Cas9 and sgRNA targeting LDLR. These cells were used again to further knock out IFNAR1 and then ATR. Electroporation of two sgRNAs simultaneously was avoided to minimize chromosome translocation. The sgRNAs were introduced in the following order to generate single, double, triple, quadruple, and quintuple KO/KD cells: PKR, OAS1, LDLR, IFNAR1, and ATR.

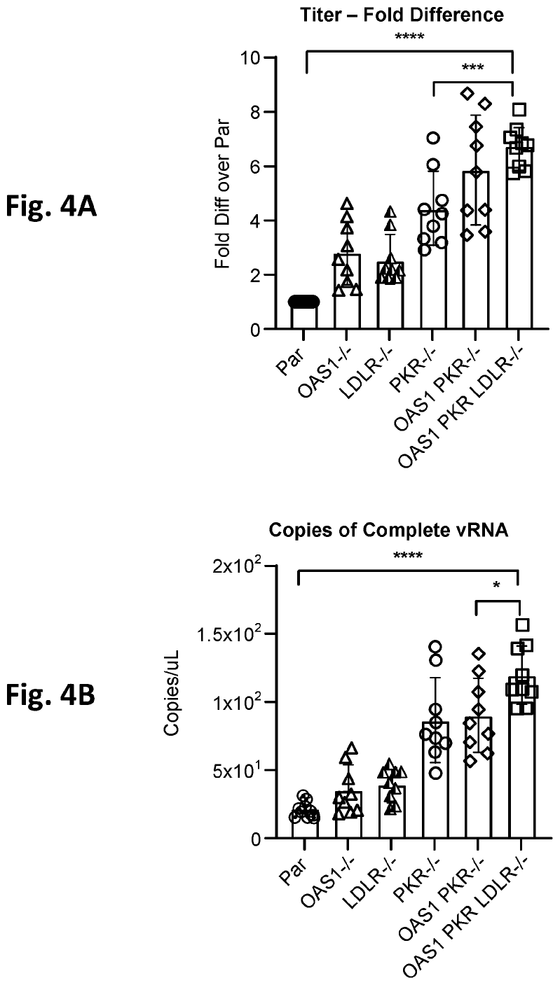

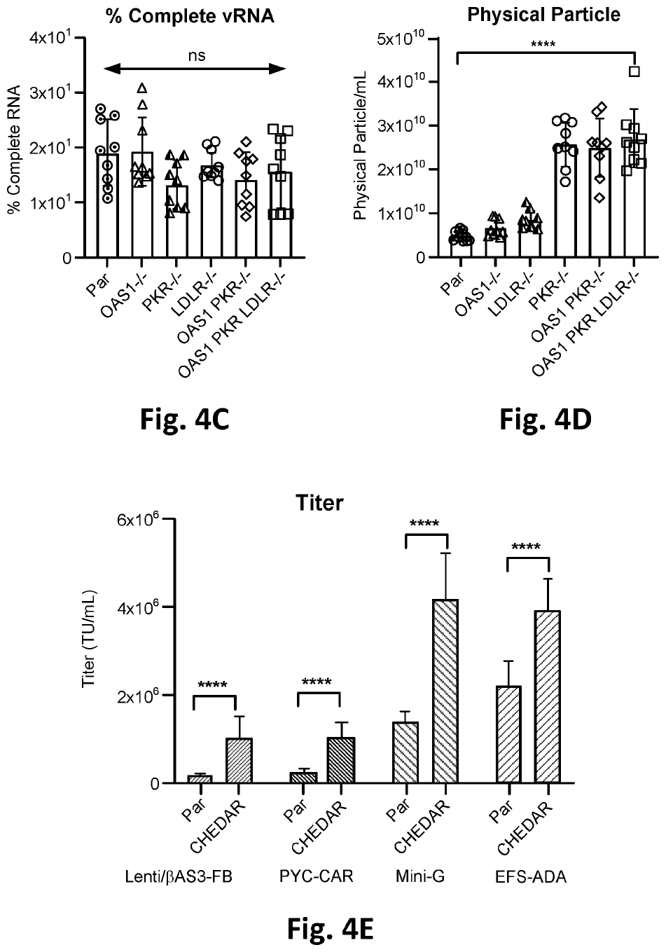

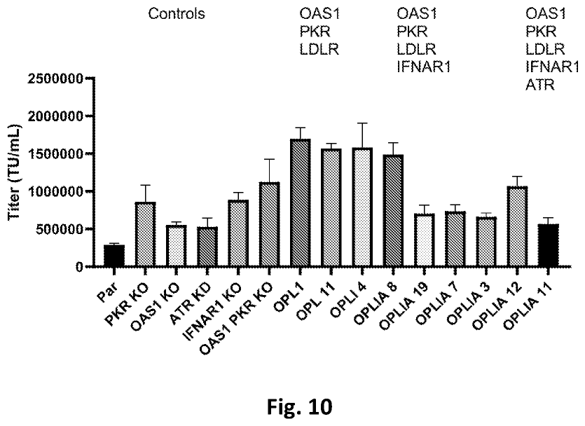

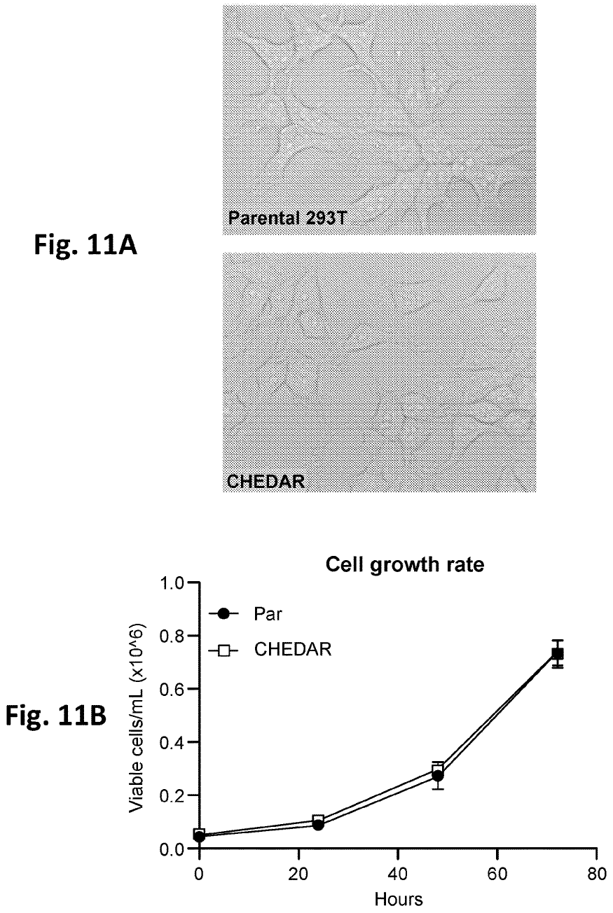

Lenti/βAS3-FB was packaged in the single-, double-, and triple-KO cells in parallel and the unconcentrated viruses titered on HT-29 cells. As shown in FIG. 4, panel A (n=9 dishes of identical cultures from three independent experiments; bars represent mean with SD; unpaired t test, *p<0.05, **p<0.01, ***p<0.001, ****p<0.0001), the OAS1 PKR double-KO cells increased titer by 5.9-±1.8-fold over the parental 293T cells. The OAS1 PKR double-KO cells slightly increased titer compared with the PKR−/− cells (4.5-±1.5-fold over the parental 293T cells). Furthermore, the OAS1 PKR LDLR triple-KO cells showed a significant increase in titer compared with the single KO cells, resulting in a 6.7-±0.7-fold increase over parental 293T cells. OAS1 PKR LDLR IFNAR1 quadruple-KO cells as well as OAS1 PKR LDLR IFNAR1 ATR quintuple-KO/KD cells did not further increase titer (FIG. 10; n=3 dishes of identical culture from one independent experiment; base represent mean with SD). These data suggest that OAS1, PKR, and LDLR triple-KO led to the highest titer, and the data were reproducible in more than one clone (FIG. 10). In addition, these edited cells shared a similar morphology and growth rate to the parental 293T cells (FIG. 11). We named the OAS PKR LDLR triple-KO cells CHEDAR.

Next, we investigated the vRNA completeness and physical particle formation of the LVs produced from these genome-engineered cells. Using the method previously described in Han et al.²⁴ to quantify complete vRNA, we observed that CHEDAR led to the highest absolute level of complete vRNA, which was significantly more than from the double-KO cells and the parental cells (FIG. 4, panel B; n=9 dishes of identical cultures from three independent experiments; bars represent mean with SD; unpaired t test, *p<0.05, **p<0.01, ***p<0.001, ****p<0.0001). None of the different genome modifications affected the percentage of RNA completeness (FIG. 3C; n=9 dishes of identical cultures from three independent experiments; bars represent mean with SD; unpaired t test, *p<0.05, **p<0.01, ***p<0.001, ****p<0.0001). CHEDAR led to a significant increase in the concentration of physical particles compared with the parental HEK293T cells but did not further increase physical particles release compared with the PKR KO alone (FIG. 4, panel D; n=9 dishes of identical cultures from three independent experiments; bars represent mean with SD; unpaired t test, *p<0.05, **p<0.01, ***p<0.001, ****p<0.0001). These data suggested that CHEDAR produced more physical particles and LVs with higher absolute complete vRNA yet a similar percentage of complete vRNA, resulting in higher titers.

To test whether the engineered packaging cell can be used to improve the titer of other LVs, we packaged four different LVs in CHEDAR cells. The vector maps are shown in FIG. 8. cPYC-CAR is an LV encoding a chimeric antigen receptor (CAR) that targets B cell maturation antigen (BCMA), an antigen present on multiple myeloma cells.²⁹ The CAR was constructed with four anti-BCMA single-chain variable fragments, fused to the CD137 (4-1BB) co-stimulatory and CD3z signaling domains under the control of an MND promoter.²⁹ Mini-G is a reverse-oriented b-globin vector with redefined enhancer element boundaries of the b-globin locus control region.⁶ The elongation factor-α gene short (EFS)-adenosine deaminase (ADA) vector consists of the human EFS promoter driving the expression of a codon-optimized human ADA gene cassette followed by the WPRE, all in the sense (forward) orientation.³⁰

Our engineered cell line was able to improve the titer of all four LVs (FIG. 4, panel E). We were able to improve the titer of Lenti/βAS3-FB by 5.9-±1.7-fold, PYC-CAR by 4.2-±0.9-fold, Mini-G by 3-±0.5-fold, and EFS-ADA by 1.8-±0.3-fold (n=9 dishes of identical cultures from three independent experiments; bars represent mean with SD; unpaired t test, *p<0.05, **p<0.01, ***p<0.001, ****p<0.0001). These data suggest that the OAS1 PKR LDLR triple-KO cells can be used to improve the packaging process for different LVs, especially LVs with low titer and LVs in reverse orientation.

Packaging with Transcription Elongation Factors Improved RNA Completeness and Titer

In our previous study, we reported that Lenti/βAS3-FB vRNA is truncated during vRNA production, and the truncated RNA cannot be reverse transcribed and integrated into the host cell genome.¹⁹ We hypothesized that packaging cells overexpressing transcription elongation factors SPT4 and SPT5 may result in more complete vRNA. SPT4 and SPT5 are components of the 5,6-Dichloro-1-b-D-ribofuranosylbenzimidazole sensitivity-inducing factor complex (DSIF complex), which regulates transcription elongation by RNA polymerase II (RNA Pol II). SPT4-SPT5 complex promotes Pol II processivity by traveling with Pol II throughout transcription elongation.³¹ Recent evidence suggests that the SPT4-SPT5 complex binds to the DNA exit region on Pol II, assisting the rewinding of DNA and preventing aberrant backtracking of Pol IL^{32,33}

To test our hypothesis, we packaged Lenti/βAS3-FB with co-transfection of SPT4/5 expression plasmids. SPT4 and SPT5 were cloned into expression plasmids under the control of an MND promoter and transiently transfected into the 293T cells with the rest of the packaging plasmids 24 h after plating the cells. Four conditions were tested: 5 μg of GFP plasmids alone, 2.5 μg of SPT4 plasmids with 2.5 μg of GFP plasmids, 2.5 μg of SPT5 plasmids with 2.5 μg of GFP plasmids, and 2.5 μg of SPT4 plasmids with 2.5 μg of SPT5 plasmid. GFP plasmids were used as fillers to ensure an equal mass of plasmids were transfected in each condition.

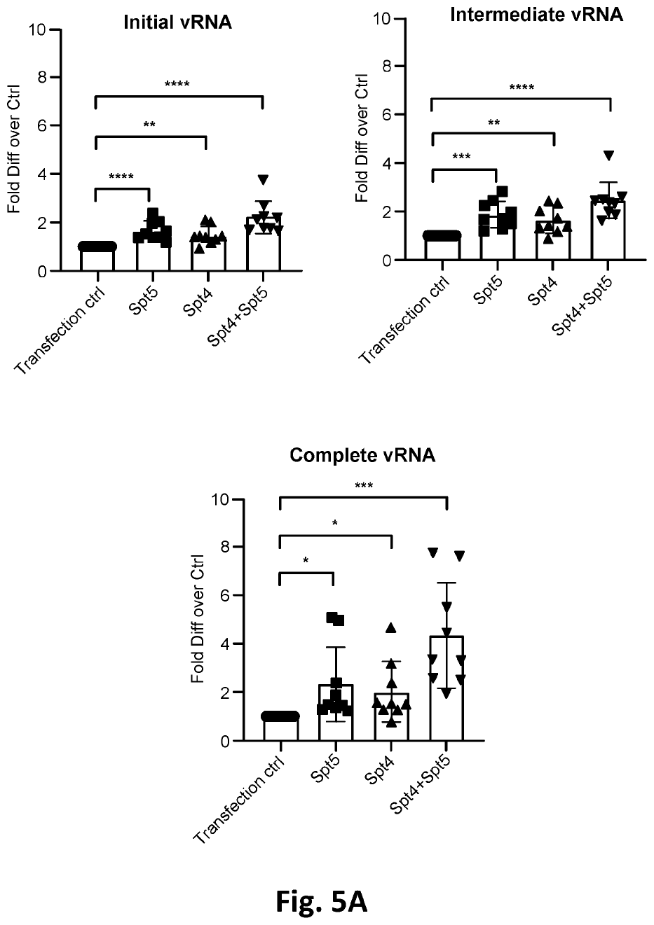

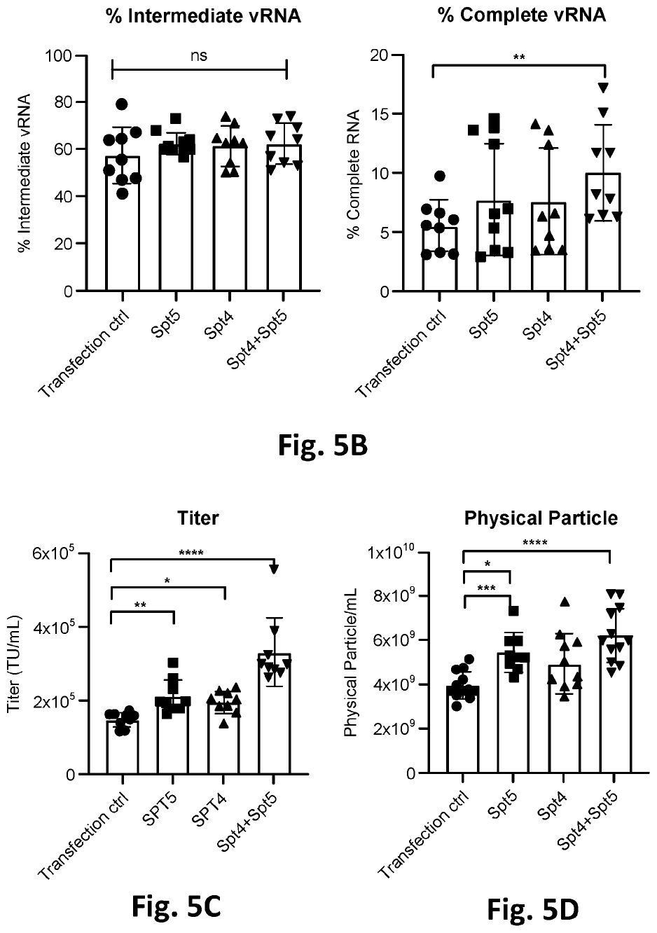

We first assessed the effect of SPT4 and SPT5 on vRNA production (FIG. 5, panel A; n=9 dishes of identical cultures from three independent experiments; bars represent mean with SD; unpaired t test, *p<0.05, **p<0.01, ***p<0.001, ****p<0.0001). Compared with the transfection control, adding SPT5 alone increased the level of initial vRNA by 1.7-±0.4-fold, the level of intermediate vRNA by 1.9-±0.5-fold, and the level of complete vRNA by 2.4-±1.5-fold; adding SPT4 alone increased the level of initial vRNA by 1.5-±0.4-fold, the level of intermediate vRNA by 1.6-±0.5-fold, and the complete vRNA by 2-±1.2-fold. These data suggest that there was a gradual enrichment in vRNA level toward the 3′ end, when the transcription elongation factors were added. Moreover, adding both SPT4 and SPT5 expression plasmids during packaging increased the level of initial vRNA by 2.2-±0.6-fold, the level of intermediate vRNA by 2.4-±0.7-fold, and the level of complete vRNA by 4.4-±2-fold compared with the transfection control. We did not observe an increase in the percentage of intermediate RNA, probably because the primers and probes to detect the initial vRNA (in RU5) and intermediate vRNA (in the primer binding site sequence) are very close to each other (FIG. 5, panel B; n=9 dishes of identical cultures from three independent experiments; bars represent mean with SD; unpaired t test, *p<0.05, **p<0.01, ***p<0.001, ****p<0.0001). Nevertheless, we indeed observed a significant increase in the percentage of complete vRNA when both SPT4 and SPT5 were added. These data suggest that SPT4 and SPT5 additively facilitates transcription elongation.

Next, we examined whether the increase in vRNA correlated with titer (FIG. 5, panel C; n=9 dishes of identical cultures from three independent experiments; bars represent mean with SD; unpaired t test, *p<0.05, **p<0.01, ***p<0.001, ****p<0.0001). Compared with the transfection control, adding SPT5 alone increased titer by 1.4-±0.2-fold, adding SPT4 alone increased titer by 1.3-±0.1-fold, and adding both SPT4 and SPT5 increased titer by 2.2-±0.4-fold.

We also observed increased levels of physical particles measured by p24 ELISA when SPT4 and SPT5 were added (FIG. 5, panel D; n=9-12 dishes of identical cultures from three to four independent experiments; bars represent mean with SD; unpaired t test, *p<0.05, **p<0.01, ***p<0.001, ****p<0.0001). This observation suggested that SPT4 and SPT5 potentially enhanced transcription elongation not only for the transfer plasmid but also the packaging and envelope plasmids, resulting in increased physical particle release.

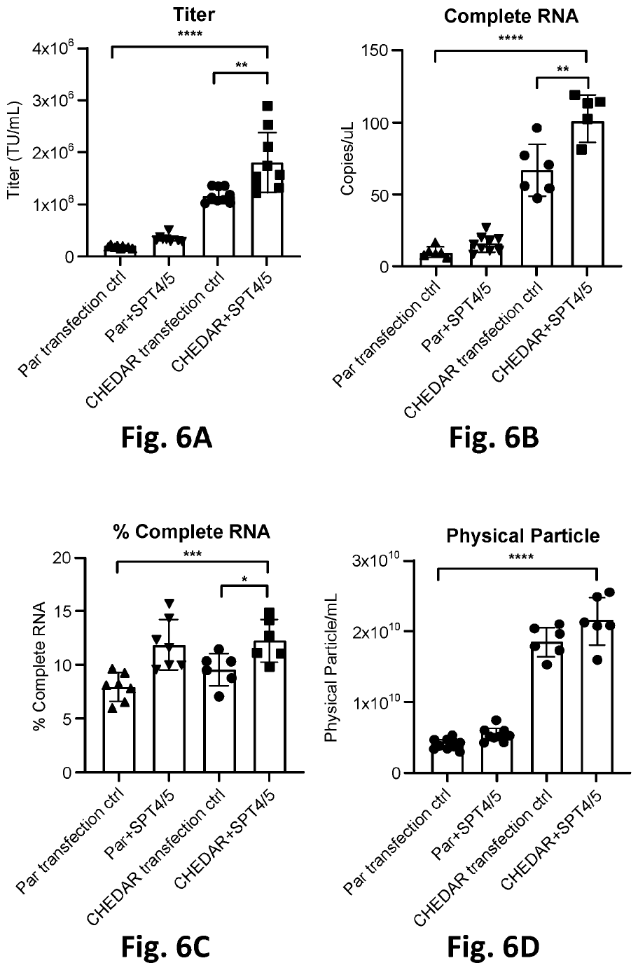

Packaging with transcription elongation factors in the CHEDAR cell line increased physical particle formation, RNA production, and titers. Finally, we explored the combinatorial effects of packaging with the transcription elongation factors in the CHEDAR cell line. As shown in FIG. 6, panel A, packaging in the parental HEK293T cells with the addition of SPT4 and SPT5 increased titer by 2-±0.4-fold compared with the parental HEK293T transfection control. Packaging in the CHEDAR cell line with the transfection control increased titer by 7-±1-fold over the parental HEK293T transfection control. Packaging in the CHEDAR cell line with the addition of SPT4 and SPT5 increased titer by 10.7-±3.2-fold over the parental HEK293T transfection control (n=9 dishes of identical cultures from three independent experiments; bars represent mean with SD; unpaired t test, *p<0.05, **p<0.01, ***p<0.001, ****p<0.0001). We also observed a significant increase in the level of complete vRNA, the percentage of complete and the level of physical particles when SPT4 and SPT5 were added to the CHEDAR cell line compared with the parental transfection control (FIG. 6, panels B-D; n=6-9 dishes of identical cultures from two to three independent experiments). Packaging with the addition of SPT4 and SPT5 in the CHEDAR cell line further improved titer and RNA production compared with the CHEDAR transfection control, suggesting that these two strategies work additively to increase titer.

### DISCUSSION

Low titer and infectivity of complex lentiviral vectors, like β-globin LVs and CAR-T LVs, create barriers for clinical and commercial applications of gene and cell therapy. We elucidated the mechanisms leading to low titer and infectivity: (1) RFs impeding various steps of the lentiviral life cycle; (2) vRNA truncations. RFs are not only IFN-dependent genes in innate immunity but also cellular proteins inhibiting lentiviral life cycle. We first conducted a targeted CRISPR-Cas9 mediated KO screen focusing on genes in the immune response, receptor-mediated virus entry, transcription factors, and DNA damage response pathway. We showed that knocking out OAS1, LDLR, and PKR in the HEK293T cells increased the titer of LVs, and knocking out all three genes in a monoclonal cell line, CHEDAR, further increased titer of Lenti/βAS3FB 4-8-fold higher than the titer of the vectors produced from the parental HEK293T cells.

LDLR serves as the major cellular entry port of VSVG-pseudotyped LVs, and other LDLR family proteins serve as the alternative, yet less effective, binding target.³⁴ One initial hypothesis of the LDLR-dependent decrease of vector titer is that LVs interact with HEK293T LDLR at the plasma membrane and re-enter the packaging cells, resulting in the loss of LVs. However, Otahal er al. showed that VSVG granules colocalized with LDLR in the ER-Golgi intermediate compartment (ERGIC) and aggresome/autophagosome.³⁵ They proposed a model that LDLR and VSVG interact prematurely in ERGIC, and these VSVG-LDLR complexes are subsequently rerouted to be degraded in aggresome/autophagosome.³⁵ We also observed that the LDLR−/− cells and the parental HEK293T cells were equally infected (data not shown), suggesting that knocking out LDLR did not prevent LVs from reentering the cells. The latter hypothesis of aggresome/autophagosome degradation of the LDLR-VSVG complex is more likely to explain the increase in titer in the LDLR−/− cells.

The 2′-5′ oligoadenylate synthetases (OASs) are a family of antiviral proteins consisting of OAS1, OAS2, OAS3, and OASL. The OAS proteins are expressed at low levels and are augmented upon IFN induction.³⁶ The OAS1, OAS2, and OAS3 proteins can be activated upon detecting double-stranded RNA (dsRNA) to produce 2′-5′ oligoadenylates (2-5As),³⁷ which subsequently activates RNase L.³⁸ RNase L degrades both cellular and vRNA, thereby inhibiting viral replication.³⁹ While OAS1-3 proteins are well known for their RNase L-dependent activity, recent evidence suggests that OAS1 can directly inhibit viral replication independently of RNase L. Kristiansen et al. reported that OAS can be released to the extracellular space and acts as a paracrine antiviral agent without activation of RNase L. In our study, we did not observe an increase in titer packaging in RNASEL KD 293T cells, suggesting that the RNase L-independent antiviral activity may explain the increase in titer with the OAS1−/− 293T cells. Future studies to explore the mechanisms leading to the titer increase in OAS1−/− cells are needed. Although the IFN signaling is uncoupled from cytokine secretion in 293T cells, we showed that some of the antiviral effectors, such as OAS1 and PKR, are still active in 293T cells, and knocking them out improved packaging efficiency.

In addition to exploring the lentiviral RFs, we overexpressed transcription elongation factors SPT4 and SPT5 to enhance the production of complete vRNA. Expressing either SPT4 or SPT5 individually led to a ˜2-fold increase in complete vRNA. SPT4 and SPT5 worked synergistically, because expressing both transcription elongation factors together led to a ˜4-fold increase in complete vRNA and a 2-fold increase in the percentage of complete vRNA. Moreover, the transcription elongation factors showed an additive effect with the CHEDAR KO cell line to further increase complete vRNA by 1.5-fold, resulting in a 1.2-fold increase in titer. These results suggest that overexpressing transcription elongation factors facilitates the vRNA production in both parental HEK293T and the triple-KO cells. Future studies are needed to explore the addition of other transcription elongation factors.

In summary, we present a new packaging cell line, CHEDAR, with OAS1, LDLR, and PKR knocked out to prevent the antiviral responses and inhibition of the lentiviral life cycle. We also showed that including transcription elongation factors SPT4 and SPT5 improved vRNA completion. Combining the cell line and packaging with the transcription elongation factors improved titer by 10.7-±3.2-fold.

### Methods

### LV Production and Titration

The LVs used in this study include EFS-ADA,30 Lenti/βAS3-FB,²³ Mini-G,6 and PYC-CAR. The LV packaging and titration protocols were previously described in Han et al.²⁴ Briefly, genetically modified and WT 293T cells were plated in six-well plates and transiently transfected with fixed amount of HIV Gag/Pol, Rev, VSV-G expression plasmids, and equimolar amounts of transfer plasmids with TransIT-293 (Mirus Bio, Madison, WI). For certain experiments, SPT4 and SPT5 expression plasmids or a non-packageable GFP plasmid were added to the transfection mix. About 20 h after transfection, transfected cells were incubated in D10 containing 10 mM sodium butyrate and 20 mM HEPES for 6-8 h. Cells were then washed with PBS and cultured in fresh D10 for approximately 40 h. Viral supernatants were collected and filtered through a 0.45-mm filter. If needed, viral supernatants were concentrated by ultracentrifugation at 26,000 rpm for 90 min. Both raw and concentrated viruses were kept at −80° C. for long-term storage.

Viral titers were determined by transducing HT-29 human colon carcinoma cells with different dilutions of the LVs and pelleted the cells ˜60 h after transduction. Genomic DNA was extracted using either the PURELINK® Genomic DNA Mini Kit (Invitrogen, Waltham, MA) or QUICKEXTRACT® DNA Extraction Solution (Lucigen, Middleton, WI). Viral titers were calculated as vector copy number (VCN) times cell number at the time of transduction times dilution factor. VCN was defined as the ratio of the copies of the HIV-1 PBS region to the copies of the SDC4 endogenous reference gene and measured by Droplet Digital PCR (ddPCR). The droplet generation process was described in Hindson et al.,⁴⁰ and the ddPCR cycling conditions and protocols were described previously in Han et al.²⁴

sgRNA Construction

sgRNAs were designed using the Benchling CRISPR online tool, and oligonucleotides were synthesized by Integrated DNA Technologies (San Diego, CA). sgRNAs were in vitro transcribed following the protocol as previously described.⁴¹ Briefly, sgRNA was assembled as DNA and PCR amplified to generate enough DNA templates. The DNA template was in vitro transcribed into sgRNAs using the HISCRIBE® T7 Quick High Yield RNA Synthesis Kit (New England Biolabs; Ipswich, MA). RNA was purified using the RNEAST® MinElute Cleanup Kit (Qiagen; Valencia, CA), following the manufacturer's protocol.

### Electroporation

At room temperature, 2×10⁵ cells per condition were pelleted at 100×g for 10 min and resuspended in 10 mL of SF solution with supplements (Lonza; Basel, Switzerland). Then 100 pmol of Cas9 was added to 240 pmol of sgRNA to form the RNP complex, and the RNP was incubated on ice for 10-20 min. The cells and RNP were combined and electroporated with the Lonza 4D Nucleofector system (program CM-130). After electroporation, cells were recovered at room temperature for 10 min and then transferred to a 24-well plate. To analyze the cutting efficiency, cells were pelleted for DNA extraction 24-48 h after electroporation using the PureLink Genomic DNA Mini Kit (Invitrogen/Thermo Fisher Scientific; Carlsbad, CA). DNA regions around the cut site were PCR amplified, and the PCR products were sent for Sanger sequencing. The cutting efficiencies were analyzed using the Synthego ICE Analysis tool (2019. v3.0). After confirming the cutting efficiency, electroporated cells were single-cell sorted at the University of California, Los Angeles (UCLA) Broad Stem Cell Research Center Flow Cytometry Core. About 2 weeks after sorting, isogenic cell clones were collected for genomic DNA extraction, PCR amplification, Sanger sequencing, and ICE analysis to identify KO or knockdown clones.

### Flow Cytometry

All flow cytometry analyses were performed on BD LSRFortessa and BD FACSAria II. The cells were harvested and washed with PBS and stained for 30 min at 4° C. or 15 min at room temperature with the anti-LDLR antibody (R&D Systems, Minneapolis, MN) and DAPI. For fluorescence-activated cell sorting (FACS) of single cells, cells were stained with DAPI, and live cells sorted into one cell per well in 96-well plates.

### Western Blot

The western blot protocol was previously described by Crowell et al.⁴³ All samples except OAS1 did not require prior treatments. To induce OAS1 expression, cells were incubated with 10 ng/mL recombinant human IFN-beta protein (R&D Systems, Minneapolis, MN) at 37° C. for 24 h prior to harvesting the cells. Then 2×10⁶ cells were collected and lysed in RIPA buffer (50 mM Tris-HCL pH8.0, 150 mM NaCl, 1% NP-40, 0.5% sodium deoxycholate, 0.1% SDS) with HALT protease inhibitor cocktail (Thermo Fisher, Waltham, MA). To determine the protein concentration, the BCA assay was conducted using the Pierce BCA Protein assay kit (Thermo Fisher, Waltham, MA) following the manufacturer's protocol. An equal amount of protein was run on NuPAGE 4%-12% Bis-Tris Gel (Novex), transferred onto polyvinylidene fluoride (PVDF) membranes (Millipore Sigma), and incubated with the primary antibodies at 4° C. overnight. The primary antibodies used in this study include anti-SPT4 (Cell Signaling Technology, product #648285, Danvers, MA), anti-SPT5 (Santa Cruz Biotechnology, sc390961, Dallas, TX), anti-ATR (Cell Signaling Technology, Product #13934, Danvers, MA), anti-IFNAR1 (Abcam, ab124764, Cambridge, MA), anti-β-Actin (Cell Signaling Technology, product #3700, Danvers, MA), anti-OAS1 (Cell Signaling Technology, product #14498, Danvers, MA), and anti-tubulin (ABCAM®, ab56676). The primary antibodies were then detected with horseradish peroxidase (HRP)-conjugated secondary antibodies followed by HRP chemiluminescence (www.ncbi.nlm.nih.gov/pmc/articles/PMC6710009/).

vRNA Analysis by ddPCR

RNA was extracted from 140 mL of unconcentrated viral supernatant using the QIAmp Viral RNA Mini Kit (Qiagen), treated with DNAse I (Invitrogen, Waltham, MA), reverse transcribed with Moloney Murine Leukemia Virus (M-MLV) reverse transcriptase (Invitrogen, Waltham, MA), as previously described in Han et al.²⁴

The abundance of initial, intermediate, and complete RNA was quantified by ddPCR using different primer and probe sets. Initial RNA was quantified by the amplification with the R/U5 primers and probe: forward primer 5′-GCTAACTAGGGAACCCACTGCT-3′ (SEQ ID NO:19), reverse primer 5′-GGGTCTGAGGGATCTCTAGTTACCA-3′ (SEQ ID NO:20), and probe 5′-FAM-CTTCAAGTAGTGTGTGCCCGTCTGT-31ABFQ-3′ (SEQ ID NO:21) (Integrated DNA Technologies). Intermediate RNA was quantified by the amplification with the PBS primers and probe: forward primer 5′-AAGTAGTGTGTGCCCGTCTG-3′ (SEQ ID NO:22), reverse primer 5′-CCTCTGGTTTCCCTTTCGCT-3′ (SEQ ID NO:23), and probe 5′-FAM-CCCTCAGACCCTTTTAGTCAGTGTGGAAAATCTCTAG-31ABFQ-3′ (SEQ ID NO:24). Complete RNA was quantified by the amplification with the U3/R primers and probe: forward primer 5′-AGCAGTGGGTTCCCTAGTTAG-3′ (SEQ ID NO:25), reverse primer 5′-GGGACTGGAAGGGCTAATTC-3′ (SEQ ID NO:26), and probe 5′-FAM-AGA-GACCCAGTACAAGCAAAAAGCAG-31ABFQ-3′ (SEQ ID NO:27). The cycling conditions were 95° C. for 10 min for one cycle (94° C. for 30 s and 60° C. for 1 min) for 40 cycles, 10 min at 98° C. for one cycle, and a 12° C. hold.

### P24 Assay

UCLA/Center for AIDS Research (CFAR) Virology Core kindly conducted all the p24 ELISA to quantify p24 antigen concentration in unconcentrated viral supernatants using the Alliance HIV-1 p24 Antigen ELISA Kit (catalog #NEK050, PerkinElmer, Waltham, MA), following the manufacturer's manual.

### Statistical Analyses

Descriptive statistics, such as a number of observations, mean, and SD were reported and presented graphically for quantitative measurements. Unpaired t tests were used to compare columns for outcome measures, such as titers, the concentration of physical particles, copies, and percentage of RNA. In the case of normality assumption violation, nonparametric Wilcoxon rank-sum tests were used. For all statistical investigations, tests for significance were two-tailed. A p value of less than 0.05 significance level was considered to be statistically significant. All statistical analyses were conducted using GraphPad Prism version 8.0 (GraphPad Software, San Diego, CA).

### REFERENCES FOR EXAMPLE 2

1. Kohn, D. B., Booth, C., Shaw, K. L., Xu-Bayford, J., Garabedian, E., Trevisan, V., et al. (2021). Autologous ex vivo lentiviral gene therapy for adenosine deaminase deficiency. N. Engl. J. Med. 384, 2002-2013, NEJMoa2027675.
2. Aiuti, A., Cattaneo, F., Galimberti, S., Benninghoff, U., Cassani, B., Callegaro, L., Scaramuzza, S., Andolfi, G., Mirolo, M., Brigida, I., et al. (2009). Gene therapy for immunodeficiency due to adenosine deaminase deficiency. N. Engl. J. Med. 360, 447-458.
3. Cartier, N., Hacein-Bey-Abina, S., von Kalle, C., Bougneres, P., Fischer, A., Cavazzana-Calvo, M., and Aubourg, P. (2010). [Gene therapy of x-linked adrenoleukodystrophy using hematopoietic stem cells and a lentiviral vector]. Bull. Acad. Natl. Med. 194, 255-264, discussion 264-8.
4. de Ravin, S. S., Wu, X., Moir, S., Anaya-O'Brien, S., Kwatemaa, N., Littel, P., Theobald, N., Choi, U., Su, L., Marquesen, M., et al. (2016). Lentiviral hematopoietic stem cell gene therapy for X-linked severe combined immunodeficiency. Sci. Transl. Med. 8, 335ra57.
5. Morgan, R. A., Ma, F., Unti, M. J., Brown, D., Ayoub, P. G., Tam, C., Lathrop, L., Aleshe, B., Kurita, R., Nakamura, Y., et al. (2020). Creating new b-globin-expressing lentiviral vectors by high-resolution mapping of locus control region enhancer sequences. Mol. Ther. Methods Clin. Dev. 17, 999-1013.
6. Morgan, R. A., Unti, M. J., Aleshe, B., Brown, D., Osborne, K. S., Koziol, C., et al. (2020). Improved titer and gene transfer by lentiviral vectors using novel, small b-globin locus control region elements. Mol. Ther. 28, 328-340.
7. Strebel, K., Luban, J., and Jeang, K. T. (2009). Human cellular restriction factors that target HIV-1 replication. BMC Med. 7, 48.
8. Kajaste-Rudnitski, A., Pultrone, C., Marzetta, F., Ghezzi, S., Coradin, T., and Vicenzi, E. (2010). Restriction factors of retroviral replication: the example of Tripartite Motif (TRIM) protein 5a and 22. Amino Acids 39, 1-9.
9. Kajaste-Rudnitski, A., and Naldini, L. (2015). Cellular innate immunity and restriction of viral infection: implications for lentiviral gene therapy in human hematopoietic cells. Hum. Gene Ther. 26, 201-209.
10. Ferreira, C. B., Sumner, R. P., Rodriguez-Plata, M. T., Rasaiyaah, J., Milne, R. S., Thrasher, A. J., Qasim, W., and Towers, G. J. (2020). Lentiviral vector production titer is not limited in HEK293T by induced intracellular innate immunity. Mol. Ther. Methods Clin. Dev. 17, 209-219.
11. Juang, Y. T., Lowther, W., Kellum, M., Au, W. C., Lin, R., Hiscott, J., and Pitha, P. M. (1998). Primary activation of interferon A and interferon B gene transcription by interferon regulatory factor 3. Proc. Natl. Acad. Sci. USA 95, 9837-9842.
12. Fonseca, G. J., Thillainadesan, G., Yousef, A. F., Ablack, J. N., Mossman, K. L., Torchia, J., and Mymryk, J. S. (2012). Adenovirus evasion of interferon-mediated innate immunity by direct antagonism of a cellular histone posttranslational modification. Cell Host Microbe 11, 597-606.
13. Ronco, L. v., Karpova, A. Y., Vidal, M., and Howley, P. M. (1998). Human papillomavirus 16 E6 oncoprotein binds to interferon regulatory factor-3 and inhibits its transcriptional activity. Genes Dev. 12, 2061-2072.
14. Levine, A. J. (2009). The common mechanisms of transformation by the small DNA tumor viruses: the inactivation of tumor suppressor gene products: p53. Virology 384, 285-293.
15. Ank, N., West, H., Bartholdy, C., Eriksson, K., Thomsen, A. R., and Paludan, S. R. (2006). Lambda interferon (IFN-1), a type III IFN, is induced by viruses and IFNs and displays potent antiviral activity against select virus infections in vivo. J. Virol. 80, 4501-4509.
16. Williams, B. R. G. (1999). PKR; a sentinel kinase for cellular stress. Oncogene 18, 6112-6120.
17. Garcia, M. A., Gil, J., Ventoso, I., Guerra, S., Domingo, E., Rivas, C., and Esteban, M. (2006). Impact of protein kinase PKR in cell biology: from antiviral to antiproliferative action. Microbiol. Mol. Biol. Rev. 70, 1032-1060.
18. Hu, P., Bi, Y., Ma, H., Suwanmanee, T., Zeithaml, B., Fry, N. J., Kohn, D. B., and Kafri, T. (2018). Superior lentiviral vectors designed for BSL-0 environment abolish vector mobilization. Gene Ther. 25, 454-472.
19. Han, J., Tam, K., Ma, F., Tam, C., Aleshe, B., Wang, X., Quintos, J. P., Morselli, M., Pellegrini, M., Hollis, R. P., et al. (2021). b-Globin lentiviral vectors have reduced titers due to incomplete vector RNA genomes and lowered virion production. Stem Cell Rep. 16, 198-211.
20. Finkelshtein, D., Werman, A., Novick, D., Barak, S., and Rubinstein, M. (2013). LDL receptor and its family members serve as the cellular receptors for vesicular stomatitis virus. Proc. Natl. Acad. Sci. USA 110, 7306-7311.
21. Amirache, F., Levy, C., Costa, C., Mangeot, P. E., Torbett, B. E., Wang, C. X., Negre, D., Cosset, F. L., and Verhoeyen, E. (2014). Mystery solved: VSV-G-LVs do not allow efficient gene transfer into unstimulated T cells, B cells, and HSCs because they lack the LDL receptor. Blood 123, 1422-1424.
22. Otahal, A., Fuchs, R., Al-Allaf, F. A., and Blaas, D. (2015). Release of vesicular stomatitis virus spike protein G-pseudotyped lentivirus from the host cell is impaired upon low-density lipoprotein receptor overexpression. J. Virol. 89, 11723-11726.
23. Romero, Z., Urbinati, F., Geiger, S., Cooper, A. R., Wherley, J., Kaufman, M. L., Hollis, R. P., de Assin, R. R., Senadheera, S., Sahagian, A., et al. (2013). b-Globin gene transfer to human bone marrow for sickle cell disease. J. Clin. Invest. 123, 3317-3330.
24. Han, J., Tam, K., Ma, F., Tam, C., Aleshe, B., Wang, X., et al. (2021). b-Globin lenti-viral vectors have reduced titers due to incomplete vector RNA genomes and lowered virion production. Stem Cell Rep. 16, 198-211.
25. Ruzankina, Y., Pinzon-Guzman, C., Asare, A., Ong, T., Pontano, L., Cotsarelis, G., Zediak, V. P., Velez, M., Bhandoola, A., and Brown, E. J. (2007). Deletion of the developmentally essential gene ATR in adult mice leads to age-related phenotypes and stem cell loss. Cell Stem Cell 1, 113-126.
26. Brown, E. J., and Baltimore, D. (2003). Essential and dispensable roles of ATR in cell cycle arrest and genome maintenance. Genes Dev. 17, 615-628.
27. Cortez, D., Guntuku, S., Qin, J., and Elledge, S. J. (2001). ATR and ATRIP: partners in checkpoint signaling. Science 294, 1713-1716.
28. Baldwin, K., Urbinati, F., Romero, Z., Campo-Fernandez, B., Kaufman, M. L., Cooper, A. R., Masiuk, K., Hollis, R. P., and Kohn, D. B. (2015). Enrichment of human hematopoietic stem/progenitor cells facilitates transduction for stem cell gene therapy. Stem Cells 33, 1532-1542.
29. Friedman, K. M., Garrett, T. E., Evans, J. W., Horton, H. M., Latimer, H. J., Seidel, S. L., Horvath, C. J., and Morgan, R. A. (2018). Effective targeting of multiple B-cell maturation antigen-expressing hematological malignances by anti-B-cell maturation antigen chimeric antigen receptor T cells. Hum. Gene Ther. 29, 585-601.
30. Carbonaro, D. A., Zhang, L., Jin, X., Montiel-Equihua, C., Geiger, S., Carmo, M., Cooper, A., Fairbanks, L., Kaufman, M. L., Sebire, N. J., et al. (2014). Preclinical demonstration of lentiviral vector-mediated correction of immunological and metabolic abnormalities in models of adenosine deaminase deficiency. Mol. Ther. 22, 607.
31. Fitz, J., Neumann, T., and Pavri, R. (2018). Regulation of RNA polymerase II processiv-ity by Spt5 is restricted to a narrow window during elongation. EMBO J. 37, e97965.
32. Bernecky, C., Plitzko, J. M., and Cramer, P. (2017). Structure of a transcribing RNA polymerase II-DSIF complex reveals a multidentate DNA-RNA clamp. Nat. Struct. Mol. Biol. 24, 809-815.
33. Ehara, H., Yokoyama, T., Shigematsu, H., Yokoyama, S., Shirouzu, M., and Sekine, S. I. (2017). Structure of the complete elongation complex of RNA polymerase II with basal factors. Science 357, 921-924.
34. Finkelshtein, D., Werman, A., Novick, D., Barak, S., and Rubinstein, M. (2013). LDL receptor and its family members serve as the cellular receptors for vesicular stomatitis virus. Proc. Natl. Acad. Sci. USA 110, 7306-7311.
35. Otahal, A., Fuchs, R., Al-Allaf, F. A., and Blaas, D. (2015). Release of vesicular stomatitis virus spike protein G-pseudotyped lentivirus from the host cell is impaired upon low-density lipoprotein receptor overexpression. J. Virol. 89, 11723-11726.
36. Baglioni, C., Minks, M. A., and Clercq, E. (1981). Structural requirements of polynucleotides for the activation of (20-50) An polymerase and protein kinase. Nucleic Acids Res. 9, 4939-4950.
37. Dong, B., and Silverman, R. H. (1997). A bipartite model of 2-5A-dependent RNase L. J. Biol. Chem. 272, 22236-22242.
38. Clemens, M. J., and Williams, B. R. G. (1978). Inhibition of cell-free protein synthesis by pppA20 p50 A20 p50 A: a novel oligonucleotide synthesized by interferon-treated L cell extracts. Cell 13, 565-572.
39. Kristiansen, H., Scherer, C. A., McVean, M., Iadonato, S. P., Vends, S., Thavachelvam, K., Steffensen, T. B., Horan, K. A., Kuri, T., Weber, F., et al. (2010). Extracellular 20-50 oligoadenylate synthetase stimulates RNase L-independent antiviral activity: a novel mechanism of virus-induced innate immunity. J. Virol. 84, 11898-11904.
40. Hindson, B. J., Ness, K. D., Masquelier, D. A., Belgrader, P., Heredia, N. J., Makarewicz, A. J., Bright, I. J., Lucero, M. Y., Hiddessen, A. L., Legler, T. C., et al. (2011). High-Throughput Droplet Digital PCR System for Absolute Quantitation of DNA Copy Number. Anal. Chem. 83, 8604-8610.
41. Crowell, P. D., Fox, J. J., Hashimoto, T., Diaz, J. A., Navarro, H. I., Henry, G. H., Feldmar, B. A., Lowe, M. G., Garcia, A. J., Wu, Y. E., et al. (2019). Expansion of luminal progenitor cells in the aging mouse and human prostate. Cell Rep. 28, 1499-1510.e6.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A recombinant retroviral packaging cell, said packaging cell comprising:
a mammalian cell wherein one or more mammalian genes that inhibit virus production are knocked out or knocked down, said one or more mammalian genes that inhibit virus production comprise
(a) a mammalian gene that encodes a low-density lipoprotein receptor (LDLR);
(b) a mammalian gene that encodes interferon alpha and beta receptor subunit 1 (IFNAR1) or interferon alpha and beta receptor subunit 2 (IFNAR2) or a combination thereof of;
(c) one or more mammalian genes that regulates the DNA damage response pathway;
(d) one or more mammalian genes that regulates transcription; or
(e) one or more mammalian genes that regulates innate immunity; or
(f) any combination of (a)-(e) above.

2. (canceled)

3. The packaging cell according to claim 1, wherein said cell is modified to provide at least two packaging components for the surface or envelope of a retrovirus, wherein optionally, said packaging cell is modified to express retroviral Gag, Pol, and Env genes.

4. (canceled)

5. (canceled)

6. (canceled)

7. (canceled)

8. The packaging cell of claim 1, wherein
said one or more mammalian genes that regulates innate immunity comprises 2′-5′-oligoadenylate synthetase 1 (OAS1), 2′-5′-oligoadenylate synthetase 2 (OAS2), 2′-5′-oligoadenylate synthetase 3 (OAS3), or 2′-5′-oligoadenylate synthetase like (OASL), or a combination thereof;
said one or more mammalian genes that regulates the DNA damage response pathway or regulates transcription comprises the PKR gene (a gene that encodes protein kinase R); or
said one or more mammalian genes that regulates the DNA damage response pathway comprises the ATR gene (a gene that encodes serine/threonine-protein kinase ATR); or
any combination thereof.

9. (canceled)

10. (canceled)

11. (canceled)

12. (canceled)

13. (canceled)

14. (canceled)

15. (canceled)

16. (canceled)

17. (canceled)

18. (canceled)

19. The packaging cell of claim 8, wherein ATR is knocked down and not knocked out.

20. (canceled)

21. The packaging cell according to claim 8, wherein said one or more mammalian genes that are knocked out or knocked down comprise a member of the OAS gene family, PKR, LDLR or any combination thereof, optionally, wherein said one or more mammalian genes that are knocked out or knocked down comprise one or more genes selected from the group consisting of OAS1, LDLR, and PKR.

22. (canceled)

23. The packaging cell of claim 21, wherein said one or more mammalian genes that are knocked out or knocked down comprise OAS1.

24. The packaging cell according to claim 21, wherein said one or more mammalian genes that are knocked out or knocked down comprise LDLR.

25. The packaging cell according to claim 21, wherein said one or more mammalian genes that are knocked out or knocked down comprise PKR.

26. (canceled)

27. (canceled)

28. (canceled)

29. (canceled)

30. (canceled)

31. (canceled)

32. The packaging cell according to claim 1, wherein said cell is further modified to express or to overexpress a transcription elongation factor.

33. The packaging cell of claim 32, wherein said transcription elongation factor is selected from SPT4 and/or SPT5.

34. (canceled)

35. The packaging cell according to claim 33, wherein an expression cassette that expresses elongation factors SPT4 and/or SPT5 is episomal in said packaging cell or is integrated into the genome of said packaging cell.

36. (canceled)

37. (canceled)

38. (canceled)

39. The packaging cell according to claim 1, wherein said mammalian cell is selected from the group consisting of HEK293, HEK293T, TE671, HT1080, 3T3, K562, 3T3, U937, and H9.

40. (canceled)

41. The packaging cell according to claim 1, wherein said cell, when transfected with a defective recombinant retroviral genome, produces complete virion at a higher titer and/or infectivity than the same cell without said one or more mammalian genes knocked out and without transcription elongation factors overexpressed.

42. The packaging cell of claim 41, wherein said packaging cell increases lentiviral vector titer, increases titer for complex lentiviral vectors, or increases titer for lentiviral vectors in reverse orientation.

43. (canceled)

44. (canceled)

45. The packaging cell according to claim 1, wherein said cell is further transfected with a defective, recombinant retroviral genome containing a nucleotide sequence of interest, wherein optionally, said nucleotide sequence of interest comprises a gene or cDNA selected from the group consisting of βAS3, FOXP3, WAS, RAG1, CAR (chimeric antigen receptor), and TCR (T-cell receptor).

46. The packaging cell of claim 45, wherein said defective, recombinant retroviral genome comprises a lentiviral (LV) genome, wherein optionally said LV genome comprises an HIV LV genome or comprises an expression cassette in reverse orientation or a combination thereof.

47. (canceled)

48. (canceled)

49. (canceled)

50. The packaging cell of claim 46, wherein said LV genome comprises Lenti/βAS3-FB.

51. A method of producing a retrovirus vector, said method comprising:
transfecting a packaging cell according to claim 1 with a defective, recombinant retroviral genome containing a nucleotide sequence of interest; and culturing said transfected packaging cell
wherein said defective, recombinant retroviral genome is packaged within a viral capsid within said cultured cell to produce a virion; and
recovering and isolating said virion.

52. The method of claim 51, wherein said defective, recombinant retroviral genome is selected from a lentiviral (LV) genome, an HIV LV genome, and a VL genome in reverse orientation.

53. (canceled)

54. The method according to claim 51, wherein said nucleotide sequence of interest comprises a gene or cDNA selected from the group consisting of βAS3, FOXP3, WAS, RAG1, CAR, and TCR.


SEQUENCE LISTING (27 sequences)

SEQ ID NO: 1 - DNA, 20 bp, Artificial
  CRISPR Guide Sequence
aaacacttcttcatggtatg

SEQ ID NO: 2 - DNA, 20 bp, Artificial
  CRISPR Guide Sequence
ctgaaggaaaggtgcttccg

SEQ ID NO: 3 - DNA, 20 bp, Artificial
  CRISPR Guide Sequence
aaagtgctagctggttgtgc

SEQ ID NO: 4 - DNA, 20 bp, Artificial
  CRISPR Guide Sequence
gtctgtcacctgcaaatccg

SEQ ID NO: 5 - DNA, 20 bp, Artificial
  CRISPR Guide Sequence
aatacataccgtcagaagca

SEQ ID NO: 6 - DNA, 20 bp, Artificial
  CRISPR Guide Sequence
cgggccgaccgcatttggga

SEQ ID NO: 7 - DNA, 20 bp, Artificial
  CRISPR Guide Sequence
ttatcctcgcagcgattgcg

SEQ ID NO: 8 - DNA, 20 bp, Artificial
  CRISPR Guide Sequence
tggaccgtgacaaccctgag

SEQ ID NO: 9 - DNA, 20 bp, Artificial
  CRISPR Guide Sequence
aaacaacaagggcccaatgg

SEQ ID NO: 10 - DNA, 20 bp, Artificial
  CRISPR Guide Sequence
cgaattcccgagtccaacca

SEQ ID NO: 11 - DNA, 20 bp, Artificial
  CRISPR Guide Sequence
gataggcaaaaaagcacgag

SEQ ID NO: 12 - DNA, 20 bp, Artificial
  CRISPR Guide Sequence
tctgctctgacattgcgccg

SEQ ID NO: 13 - DNA, 20 bp, Artificial
  CRISPR Guide Sequence
gtacctgagtcaacttcagg

SEQ ID NO: 14 - DNA, 20 bp, Artificial
  CRISPR Guide Sequence
ggtcactgcgacgttcacag

SEQ ID NO: 15 - DNA, 20 bp, Artificial
  CRISPR Guide Sequence
gatctgagatgagctctctc

SEQ ID NO: 16 - DNA, 20 bp, Artificial
  CRISPR Guide Sequence
gaagaagttggtcttgaagg

SEQ ID NO: 17 - DNA, 20 bp, Artificial
  CRISPR Guide Sequence
gcaactattcgcagtccgag

SEQ ID NO: 18 - DNA, 20 bp, Artificial
  CRISPR Guide Sequence
ctgtcctaaaaccagaagct

SEQ ID NO: 19 - DNA, 22 bp, Artificial
  PCR primer
gctaactagggaacccactgct

SEQ ID NO: 20 - DNA, 25 bp, Artificial
  PCR primer
gggtctgagggatctctagttacca

SEQ ID NO: 21 - DNA, 25 bp, Artificial
  PCR probe
cttcaagtagtgtgtgcccgtctgt

SEQ ID NO: 22 - DNA, 20 bp, Artificial
  PCR primer
aagtagtgtgtgcccgtctg

SEQ ID NO: 23 - DNA, 20 bp, Artificial
  PCR primer
cctctggtttccctttcgct

SEQ ID NO: 24 - DNA, 37 bp, Artificial
  PCR probe
ccctcagacccttttagtcagtgtggaaaatctctag

SEQ ID NO: 25 - DNA, 21 bp, Artificial
  PCR primer
agcagtgggttccctagttag

SEQ ID NO: 26 - DNA, 20 bp, Artificial
  PCR primer
gggactggaagggctaattc

SEQ ID NO: 27 - DNA, 26 bp, Artificial
  PCR probe
agagacccagtacaagcaaaaagcag